(19) United States
(12) Patent Application Publication
(10) Pub. No.: US 2009/0203634 A1
(43) Pub. Date: Aug. 13, 2009

(54) Compositions and Uses of Amooranin Compounds

(75) Inventors: Cheppail Ramachandran, Miami, FL (US); P.K. Raveendran Nair, Miami, FL (US); Steven J. Melnick, Miami, FL (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: Variety Children's Hospital d/b/a Miami Children's Hospital, Miami, FL (US)

(21) Appl. No.: 11/911,471

(22) PCT Filed: Apr. 14, 2006
(86) PCT No.: PCT/US2006/013902

§ 371 Date: Aug. 4, 2008

Related U.S. Application Data
(63) Continuation-in-part of application No. 11/106,841, filed on Apr. 15, 2005.

(51) Int. Cl. A61K 31/19; C07C 61/29; C07C 69/753; C07C 255/47; A61K 31/215; A61K 31/704; A61P 35/00; C12N 5/00

(52) U.S. Cl. 514/33; 562/498; 560/116; 558/429; 514/557; 514/561; 514/547; 435/375

(57) ABSTRACT

Amooranin (AMR) has been found to cause tumor cell death through G₂/M cell cycle arrest, caspase activation, and apoptosis. Furthermore, it has been demonstrated that AMR is a substrate for P-glycoprotein. Based on these activities, AMR compounds, including AMR analogs, can be used in the treatment of a number of diseases in which aberrant cellular proliferation occurs such as drug-sensitive and drug-resistant cancers, autoimmune disorders, and inflammatory diseases.

### CROSS-REFERENCE TO RELATED APPLICATIONS

The present application is a continuation-in-part of U.S. application Ser. No. 11/106,841, filed Apr. 15, 2005, which claims benefit of U.S. Provisional Application Ser. No. 60/562,732, filed Apr. 16, 2004, each of which is hereby incorporated by reference herein in its entirety, including any figures, tables, nucleic acid sequences, amino acid sequences, and drawings.

### BACKGROUND OF THE INVENTION

In searching for new biologically active compounds, it has been found that some natural products and organisms are potential sources for chemical molecules having useful biological activity of great diversity. Plant derived cancer drugs such as taxol, podophyllotoxin, vinblastine and vincristine, are important anti-mitotic drugs, currently used for the treatment of human malignancies. These lead compounds have been used as models for the development of novel anticancer agents (Cragg, G. M. “Role of plants in the National Cancer Institute drug discovery and development program” In Human Medicinal Agents from plants, ACS Symposium Series 534, Eds. Inghom, A. D. & Balandrin, M. F., 149-169, American Chemical Society Books, Washington, D.C., 1993; Wall, M. E. and Wani, M. C. “Camptothecin and analogues: Synthesis, biological in vitro and in vivo activities and clinical possibilities” In human medicinal agents from plants, ACS symposium series 534 Eds. Kinghom, A. D & Balandrin, M. F., 149-169, American Chemical Society Books, Washington, D.C., 1993).

Taxol, a diterpene isolated from several species of yew trees, is a mitotic spindle poison that stabilizes microtubules and inhibits their depolymerization to free tubulin (Fuchs, D. A., R. K. Johnson (1978) Cancer Treat. Rep. 62:1219-1222; Schiff, P. B., J. Fant, S. B. Horwitz (1979) Nature (London) 22:665-667). Taxol is also known to have antitumor activity and has undergone a number of clinical trials which have shown it to be effective in the treatment of a wide range of cancers (Rowinski, E. K., R. C. Donehower (1995) N. Engl. J. Med. 332:1004-1014). See also, e.g., U.S. Pat. Nos. 5,157,049; 4,960,790; and 4,206,221.

Information on the molecular mechanism of drug action is quite essential for the development of any potential drug for chemotherapy. In this context, several cancer drugs have been shown to induce apoptosis in tumor cells. Apoptosis has been characterized biochemically by the cleavage of DNA into nucleosomal size fragments of 180-200 base pairs or multiples thereof, which can be detected as DNA ladder by gel electrophoresis (Skladanowski, A. and Konopa, J. Biochem Pharmacol, 1993, 46:375-382; Ohmori, T. et al. Biochem Biophys Res Commun, 1993, 192:30-36; Ling, Y-H. et al. Cancer Res, 1993, 53:1845-1852; Kolber, M. A. et al. FASEB J, 1990, 4:3021-3027). Several cancer drugs also cause a G₂/M arrest before tumor cells undergo apoptosis (Klucer, J. and Al-Rubeai, M. FEBS Lett, 1997, 400:127-130). Pro-apoptotic genes are activated and anti-apoptotic genes are suppressed during the process of cell death, leading to the activation of caspases and nucleases that serve to degrade protein and genomic DNA, respectively, within the cell (Green, D. Cell, 1998, 94:695-698).

Caspases are aspartate-specific cysteine proteases, existing as latent intracellular zymogens (Earnshaw, W. C. et al. Annu Rev Biochem, 1999, 68:383-424; Wolf, B. B. and Green, D. R. J Biol Chem, 1999, 274:20049-20052). Once activated by apoptotic signals, they can systematically dismantle the cell by cleaving key cellular and nuclear proteins with defined substrate specificities (Martin, D. S. et al. Cancer Res, 2000, 60:6776-6783; Budihardjo, I. et al. Annu Rev Cell Dev Biol, 1999, 15:269-290). Based on the sequence of action in apoptosis signaling, more than 14 caspases have been identified and organized into apoptotic initiator and processor caspases (Earnshaw, W. C. et al. Annu Rev Biochem, 1999, 68:383-424; Wolf, B. B. and Green, D. R. J Biol Chem, 1999, 274:20049-20052; Martin, D. S. et al. Cancer Res, 2000, 60:6776-6783; Budihardjo, I. et al. Annu Rev Cell Dev Biol, 1999, 15:269-290).

Terpenoids, biosynthesized in plants by the cyclization of squalene, are reported to have anti-carcinogenic and anti-inflammatory properties (Huang, M. T. et al. Cancer Res, 1994, 54:701-708; Nishino, H. Cancer Res, 1988, 48:5210-5215). Terpenoids have been shown to inhibit proliferation and induce apoptosis in tumor cells (Konopleva, M. et al. Blood, 2002, 99:326-335; Kim, D. K. et al. Int J Cancer, 2000, 87:629-636; Hoernlein, R. F. et al. J Pharmacol Exp Ther, 1999, 288:613-619; Stadheim, T. A. et al. J Biol Chem, 2002, 277:16448-16455).

The synthetic triterpenoid 2-cyano-3,12-dioxoolean-1,9-dien-28-oic acid (CDDO), and its methyl ester (CDDO-Me), have been reported to have anti-proliferative, anti-inflammatory and differentiating effects (Konopleva, M. et al Blood, 2002, 99:326-335; Stadheim, T. A. et al. J Biol Chem, 2002, 277:16448-16455; Suh, N. et al. Cancer Res, 1999, 59:336-341). Both these synthetic triterpenoids induce apoptosis by activation of caspase-8, caspase-3 and induction of mitochondrial cytochrome c release in leukemic cell lines and osteosarcoma cells (Ito, Y. et al. Cell Growth Difer, 2000, 11:261-267; Ito, Y. et al. Mol Pharinacol, 2001, 59:1094-1099).

The success of chemotherapy for the treatment of various cancers can be substantially negated through cellular mechanisms which have evolved to enable neoplastic cells to subvert the cytotoxic effects of the drug. Some cells have developed mechanisms that confer resistance to a number of structurally unrelated drugs. This multi-drug resistance (or MDR) phenomenon may arise through a number of different mechanisms.

One of the major factors contributing to MDR is the decreased drug accumulation as a result of increased efflux mediated by overexpression of P-glycoprotein (P-gp). P-gp acts as a membrane-bound ATP-dependent efflux pump that is believed to transport a variety of structurally and functionally unrelated drugs through and out the cell membrane, resulting in a wide spectrum of cross resistance (Gottesman, M. M. et al. Ann Rev Biochem, 1993, 62:385-427; Moscow, J. A. and Cowan, K. H. J. Natl Cancer Inst, 1988, 80:14-20; Biedler, J. L. Cancer, Supplement, 1992, 70:1799-1809).

A large number of drugs that can inhibit the function of P-gp have been identified and have been termed as efflux blockers, chemosensitizers, or MDR modulators (Ford, J. M. Eur J Cancer, 1996, 32A:991-1001; Sikic, B. I. Semin Oncol, 1997, 34:40-47). Clinical trials have been performed in humans to test these drugs for pharmacological inhibition of clinical MDR (Lum, B. L. et al. Cancer, 1993, 72:3502-3514; Bates, S. F. et al. Novartis Found Symp, 2002, 243:83-102). Many of these modulators have been toxic or ineffective in vivo; therefore, the identification of potential novel agents is imperative for overcoming clinical MDR in refractory patients. Tsuruo et al. (Cancer Res, 1989, 41:1967-1972) was the first to report on the pharmacological reversal of MDR with verapamil and trifluoperazine that modulated the antiproliferative activity of vincristine and increased cellular vincristine accumulation in a multidrug resistant murine leukemia cell line. Since then, numerous compounds have been shown to antagonize MDR in a variety of tissue culture assays and animal tumor models when co-administered with the chemotherapeutic agents to which the cells are resistant (Tsuruo, T., et al. Cancer Res, 1989, 41:1967-1972; Ford, J. M. and Hait, W. N. Pharmacol Rev, 1990, 42:156-199; Ford, J. M. Hematol Oncol Clin North Amer, 1995, 9:337-355).

Generally, agents that are used to antagonize MDR meliorate the drug accumulation problem present in multidrug resistant cells, but exhibit little or no effect in drug-sensitive cells. The primary mechanism by which most modulators are believed to antagonize MDR is through direct inhibition of drug efflux mediated by P-gp, resulting in restoration of cytotoxic drug accumulation in multidrug resistant cells. However, conflicting evidence exists with regard to functional similarity of different classes of modulators, the number and position of binding sites of modulators on P-gp, and also the overlapping nature of binding sites of modulators with that of cytotoxic substances.

One of the herbal preparations used in the Indian Ayurvedic system of medicine for the treatment of human malignancies contains Amoora rohituka stem bark, Semicarpus anacardium fruits and Glyzirrhiza glabra roots, and is marketed as “CANARIB” in India (Prasad, G. C. J Res Ayurveda Sidha, 1987, 8:147-167; Prasad, G. C. J Res Ayurveda Sidda, 1994, 8:147-157). Amoora rohituka is a wild tropical medicinal tree indigenous to India, Malaysia and Sri Lanka and is known as “rohera” in India. The stem bark and the seeds of the plant have been used indigenously as medicines to treat diseases of the spleen, liver, and abdomen, including cancer (Chopra R. N. et al, Glossary of Indian Medicinal Plants, Council of Scientific and Industrial Research: New Delhi, 1956; Wealth of India, Raw Materials, Vol. I, Council of Scientific and Industrial Research: New Delhi, 1973; Prasad, 1987; Prasad, 1994). In previous studies, it has been demonstrated that various extracts and triterpene acids derived from the Amoora rohituka stem bark exhibit cytotoxicity against MCF-7 human mammary adenocarcinoma cells (Rabi T. et al., Indian J Pharm Sci, 1994, 56:136-137), Dalton's lymphoma ascites cells inoculated into the peritoneal cavity of recipient mice (Rabi, T. and Gupta, R. C. Int J Pharmacogn, 1995, 33:359-361), N-nitrosomethyl urea-induced mammary adenocarcinoma in rat tumor models (Rabi T. et al., Curr Sci, 1996, 70:80-81), and other human tumor cell lines (Rabi T. et al., Phytother Res, 2002, 16:S84-S86).

The prevention and control of inflammation is also of great importance for the treatment of humans and animals. Much research has been devoted to development of compounds having anti-inflammatory properties. Certain methods and chemical compositions have been developed which aid in inhibiting or controlling inflammation, but additional anti-inflammatory methods and compositions are needed.

Immunomodulation is a developing segment of immunopharmacology. Immunomodulator compounds and compositions, as the name implies, are useful for modulating or regulating immunological functions in humans or animals. Immunomodulators may be immunostimulants for building up immunities to, or initiate healing from, certain diseases and disorders. Conversely, immunomodulators may be immunoinhibitors or immunosuppressors for preventing undesirable immune reactions of the body to foreign materials, or to prevent or ameliorate autoimmune reactions or diseases.

Immunomodulators have been found to be useful for treating systemic autoimmune diseases, such as lupus erythematosus and diabetes, as well as immunodeficiency diseases. Further, immunomodulators may be useful for immunotherapy of cancer or to prevent rejections of foreign organs or other tissues in transplants, e.g., kidney, heart, or bone marrow.

Various immunomodulator compounds have been discovered, including FK506, muramylic acid dipeptide derivatives, levamisole, niridazole, oxysuran, flagyl, and others from the groups of interferons, interleukins, leukotrienes, corticosteroids, and cyclosporins. Many of these compounds have been found, however, to have undesirable side effects and/or high toxicity. New immunomodulator compounds are therefore needed to provide a wider range of immunomodulator function.

### BRIEF SUMMARY OF THE INVENTION

In one aspect, the subject invention provides an isolated compound having the structure shown in FIG. 1 (also referred to herein as amooranin, 25-hydroxy-3-oxoolean-12-en-28-oic acid, or AMR), or a pharmaceutically acceptable salt or analog thereof. The AMR compounds of the invention are capable of reducing multidrug resistance (MDR) at low concentrations and inducing apoptosis and cytotoxicity at higher concentrations. Amooranin is a triterpene acid obtainable from Amoora rohituka stem bark that exhibits excellent anticancer properties. The principal mechanism of tumor cell death is through G₂/M cell cycle arrest, caspase activation, and apoptosis.

Another aspect of the subject invention provides methods for the control of cellular proliferation, and inducement of G₂/M cell cycle arrest, caspase activation, and apoptosis in cancer cells, including cancer cells that are resistant to chemotherapeutic agents. It is demonstrated herein that AMR is a substrate for P-glycoprotein (P-gp) and has chemosensitizing effect on doxorubicin (DOX) cytotoxicity. In both multidrug resistant leukemia (CEM/VLB) and colon carcinoma (SW620/Ad-300) cell lines, the combination of AMR and DOX showed modulation of DOX cytotoxicity. In one embodiment, an effective amount of at least one AMR compound (AMR, or a pharmaceutically acceptable salt or analog thereof) is administered to a patient.

A further aspect of the subject invention pertains to the immunosuppressive use of at least one AMR compound (AMR, or a pharmaceutically acceptable salt or analog thereof).

In another aspect, the subject invention provides pharmaceutical compositions comprising at least one AMR compound (AMR, or a pharmaceutically acceptable salt or analog thereof) and a pharmaceutically acceptable carrier.

A further aspect of the subject invention provides a process of obtaining amooranin from Amoora rohituka plant material.

### BRIEF DESCRIPTION OF THE DRAWINGS

FIG. 1 shows the chemical structure of amooranin (25-hydroxy-3-oxoolean-12-en-28-oic acid).

FIGS. 2A-2D show the effect of AMR on DOX cytotoxicity in human leukemia (CEM, CEM/VLB) and colon carcinoma (SW620, SW620/Ad-300) cell lines. In the DOX+AMR combination treatment, cells were treated with varying concentrations of DOX (μg/ml) and 1 μg/ml AMR. The percentage of surviving cells (% of control) was plotted against DOX concentration in the combination treatment. Note the modulation of DOX cytotoxicity with AMR in CEM/VLB and SW620/Ad-300 cell lines.

FIG. 3 shows DNA distribution histograms of SW620, SW620/Ad-300, CEM and CEM/VLB cells treated with 0-2.5 μg/ml AMR for 48 hours. AMR induces G₂+M arrest in all cell lines (closed arrows) and percentage of cells in G₂+M phase increased in a dose-dependent manner. Open arrows indicate SubG₀-G₁ population induced by AMR.

FIGS. 4A-4D shows the effect of AMR on cellular DOX accumulation. Human colon carcinoma (SW620, SW620/Ad-300) and leukemia cell lines were incubated with DOX (2 μg/ml), DOX (2 μg/ml)+AMR (1 μg/ml), or DOX (2 μg/ml)+AMR (2 μg/ml) for 2 hours at 37° C. Cellular DOX fluorescence was analyzed in a flow cytometer. AMR enhanced cellular DOX accumulation in multidrug resistant CEM/VLB and SW620/Ad-300 cells.

FIGS. 5A-5D show flow cytometric analysis of P-gp in human colon carcinoma and leukemia cell lines using MRK16 Mab.

FIGS. 6A and 6B show inhibition of [³H]-azidopine photoaffinity labeling of P-gp by AMR and AMR+ DOX combination in human colon carcinoma (FIG. 6A: CEM/VLB) and leukemia (FIG. 6B: SW620/Ad-300) cell lines. Lane 1, CEM/VLB or SW 620/Ad-300 with [³H]-azidopine alone; lane 2, CEM/VLB or SW620/Ad-300 with 1 μg/ml AMR; lane 3, CEM/VLB or SW620/Ad-300 with 2 μg/ml AMR; lane 4, CEM/VLB or SW620/Ad-300 with 4 μg/ml AMR; lane 5, CEM/VLB or SW620/Ad-300 with 2 μg/ml DOX+4 μg/ml AMR.

FIG. 7 shows the effect of AMR on the growth of MCF-7, MCF-7/TH and MCF-10A cells. Data points are mean±SD estimates from three independent experiments performed in triplicate. AMR IC₅₀ value of MCF-7 cell line is significantly different from that of MCF-10A and MCF-7/TH cell lines (p<0.05).

FIG. 8 shows that AMR induces G₂/M phase arrest and apoptosis. MCF-7, MCF-7/TH and MCF-10A cells were treated with the 0-2.5 μg/ml AMR for 48 hours at 37° C. The cells were stained with propidium iodide and analyzed for cell cycle distribution by DNA flow cytometry.

FIGS. 9A-9D show results of a TUNEL assay demonstrating dose-dependent induction of apoptosis by AMR. MCF-7, MCF-7/TH and MCF-10A cells were treated with 0, 1, 2, 4 and 8 μg/ml concentrations of AMR for 48 hours (FIGS. 9A-9C). After labeling with FITC-conjugated deoxynucleosides in the presence of terminal transferase, cells were analyzed for DNA fragmentation by flow cytometry. Percentage of apoptotic cells induced by AMR is plotted against drug concentrations (FIG. 9D). Results are mean±S.D. values.

FIGS. 10A-10C show DNA fragmentation induced by AMR. MCF-7 cells were treated for 48 hours with 0, 1, 2, 4 and 8 μg/ml AMR (lanes 2-6) (FIG. 10A). MCF-7/TH cells were treated for 48 hours with 0, 1, 2, 4 and 8 μg/ml AMR (lanes 2-6) (FIG. 10B). MCF-10A cells were treated for 48 hours with 0, 1, 2, 4 and 8 μg/ml AMR (lanes 2-6) (FIG. 10C). Lane 1 in A, B and C, φX174 DNA marker digested with Hae III.

FIGS. 11A-1, 11A-2, and 11B show results of flow cytometric analysis of caspase-8 activity induced by AMR. MCF-7, MCF-7/TH and MCF-10A cells were treated with the 0, 1, 2, 4 and 8 μg/ml concentrations of AMR for 48 hours. After staining with FAM-LETD-FMK and propidium iodide, cells were analyzed for caspase-8 activity (FIGS. 11A-1 and 11A-2). Percentage of caspase-8 positive cells induced by AMR treatment is plotted against drug concentrations (FIG. 11B). Results are mean±S.D. values.

FIG. 12 shows the effect of intraperitoneal infection of AMR on tumor growth rate in human tumor xenografts, compared to doxorubicin (DOX) and saline (control) infection.

FIG. 13 shows a formula (chemical structure (II)) representing AMR analogs of the invention.

FIGS. 14A-14I show AMR analogs of the invention (structures (III), (IV), (V), (VI), (VII), (VIII), (IX), (X), and (XI)), respectively.

### DETAILED DESCRIPTION OF THE INVENTION

By inhibiting the growth of cells proliferating in an aberrant manner, the therapeutic methods, compounds, and compositions of the present invention can be used to treat a number of cell proliferation disorders, such as cancers, including, but not limited to, leukemias and lymphomas, such as acute lymphocytic leukemia, acute non-lymphocytic leukemias, chronic lymphocytic leukemia, chronic myelogenous leukemia, Hodgkin's Disease, non-Hodgkin's lymphomas, and multiple myeloma, childhood solid tumors such as brain tumors, neuroblastoma, retinoblastoma, Wilms' Tumor, bone tumors, and soft-tissue sarcomas, common solid tumors of adults such as lung cancer, colon and rectum cancer, breast cancer, prostate cancer, urinary cancers, uterine cancers, bladder cancers, oral cancers, pancreatic cancer, melanoma and other skin cancers, stomach cancer, ovarian cancer, brain tumors, liver cancer, laryngeal cancer, thyroid cancer, esophageal cancer, and testicular cancer. The methods of the subject invention can be carried out in vivo or in vitro, to inhibit the growth of cancerous cells in humans and non-human mammals.

The therapeutic methods of the present invention can be advantageously combined with at least one additional therapeutic method, including but not limited to chemotherapy, radiation therapy, or any other therapy known to those of skill in the art for the treatment and management of proliferation disorders (e.g., cancer), such as administration of an anti-cancer agent.

While AMR and AMR analogs can be administered as isolated compounds, it is preferred to administer these compounds as a pharmaceutical composition. The subject invention thus further provides pharmaceutical compositions comprising AMR, or an analog thereof, as an active agent, or physiologically acceptable salt(s) thereof, in association with at least one pharmaceutically acceptable carrier. The pharmaceutical composition can be adapted for various routes of administration, such as enteral, parenteral, intravenous, intramuscular, topical, subcutaneous, and so forth. Administration can be continuous or at distinct intervals, as can be determined by a person of ordinary skill in the art.

The AMR compounds of the subject invention can be formulated according to known methods for preparing pharmaceutically useful compositions. Formulations are described in a number of sources which are well known and readily available to those skilled in the art. For example, Reminigton's Pharmaceutical Science (Martin, E. W., 1995, Easton Pa., Mack Publishing Company, 19^{th} ed.) describes formulations which can be used in connection with the subject invention. Formulations suitable for administration include, for example, aqueous sterile injection solutions, which may contain antioxidants, buffers, bacteriostats, and solutes that render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the compositions of the subject invention can include other agents conventional in the art having regard to the type of formulation in question.

The AMR compounds of the present invention include all hydrates and salts of AMR, or of AMR analogs, that can be prepared by those of skill in the art. Under conditions where the compounds of the present invention are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, alpha-ketoglutarate, and alpha-glycerophosphate. Suitable inorganic salts may also be formed, including hydrochloride, sulfate, nitrate, bicarbonate, and carbonate salts.

Pharmaceutically acceptable salts of AMR may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

As used herein, the term “analogs” refers to compounds which are substantially the same as another compound but which may have been modified by, for example, adding side groups, oxidation or reduction of the parent structure. Analogs of AMR can be readily prepared using commonly known standard reactions. These standard reactions include, but are not limited to, hydrogenation, alkylation, acetylation, and acidification reactions. Chemical modifications can be accomplished by those skilled in the art by protecting all functional groups present in the molecule and deprotecting them after carrying out the desired reactions using standard procedures known in the scientific literature (Greene, T. W. and Wuts, P. G. M. “Protective Groups in Organic Synthesis” John Wiley & Sons, Inc. New York. 3rd Ed. pg. 819, 1999; Honda, T. et al Bioorg Med. Chem. Lett., 1997, 7:1623-1628; Honda, T. et al. Bioorg. Med. Chem. Lett., 1998, 8:2711-2714; Konoike, T. et al. J. Org. Chen., 1997, 62:960-966; Honda, T. et al. J. Med. Clzem., 2000, 43:4233-4246; each of which are hereby incorporated herein by reference in their entirety). Analogs exhibiting the desired biological activity (such as reduction of multidrug resistance, induction of apoptosis, and/or cytotoxicity) can be identified or confirmed using cellular assays or other in vitro or in vivo assays, such as those disclosed herein. For example, assays that detect G₂/M cell cycle arrest, caspase activation, and/or reduction of tumor growth may be utilized.

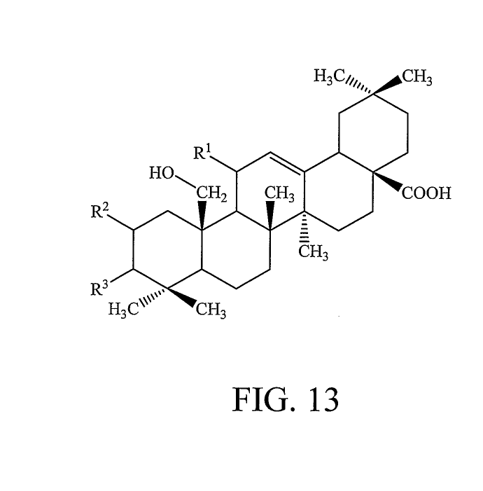

FIG. 13 shows a formula representing the chemical structure (II) of AMR analogs of the invention, wherein R¹, R², and R³ may be the same or different, and can be H, O, CN, CH₃COO, alkyl, alkenyl, alkynyl, halogen, or alkoxy.

As used in this specification, alone or in combination, the term “alkyl” refers to a straight or branched chain alkyl moiety having from one to six carbon atoms, including for example, methyl, ethyl, propyl, isopropyl, butyl, tert-butyl, pentyl, hexyl and the like.

The term “alkenyl” refers to a straight or branched chain alkyl moiety having two to six carbon atoms and having in addition one double bond, of either E or Z stereochemistry where applicable. The term alkenyl includes for example, vinyl, 1-propenyl, 1- and 2-butenyl, 2-methyl-2-propenyl and the like.

The term “alkynyl” refers to a straight or branched chain alkyl moiety having two to six carbon atoms and having in addition one triple bond. The term alkynyl includes for example, ethynyl, 1-propynyl, 1- and 2-butynyl, 1-methyl-2-butynyl and the like.

The term “alkoxy” refers to an alkyl-O-group, in which the alkyl group is as previously described. Thus, “alkoxy” includes a strain chain or branched alkoxy group containing a maximum of six carbon atoms, such as methoxy, ethoxy, propoxy, isopropoxy, butoxy, tert-butoxy, and the like.

The term “halogen” means fluorine, chlorine, bromine, or iodine.

The invention contemplates the exclusion of any substituent (selected from the group consisting of H, O, CN, CH₃COO, alkyl, alkenyl, alkynyl, halogen, and alkoxy) of any R group (R¹, R², and/or R³).

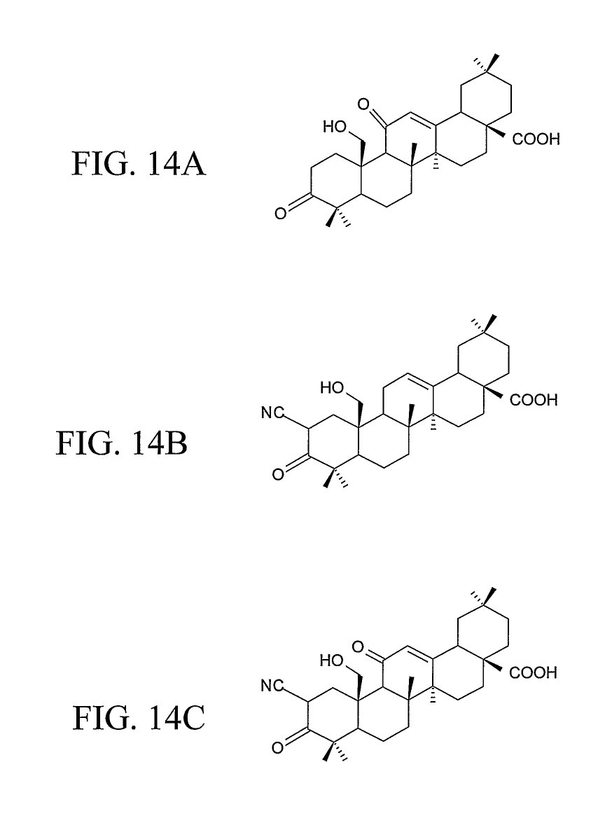

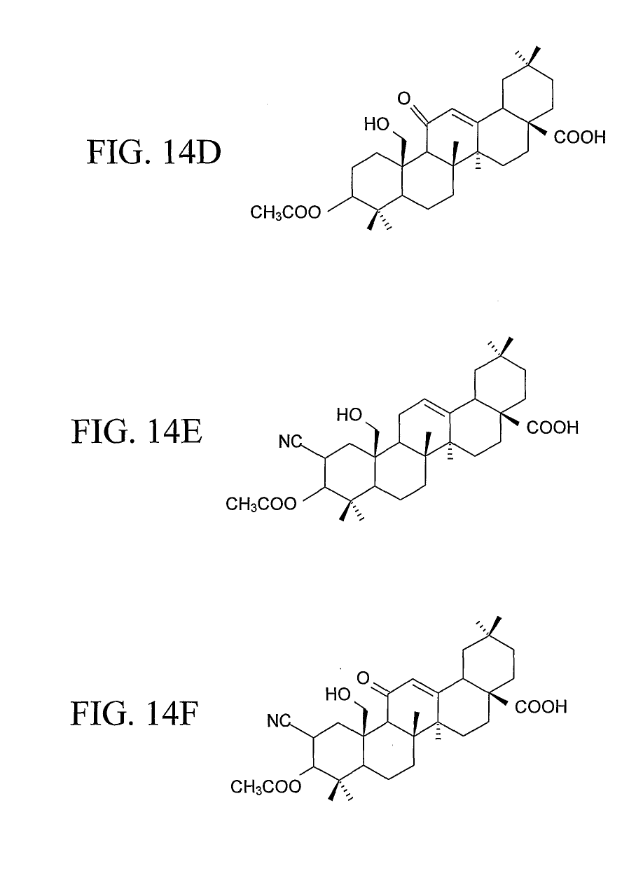

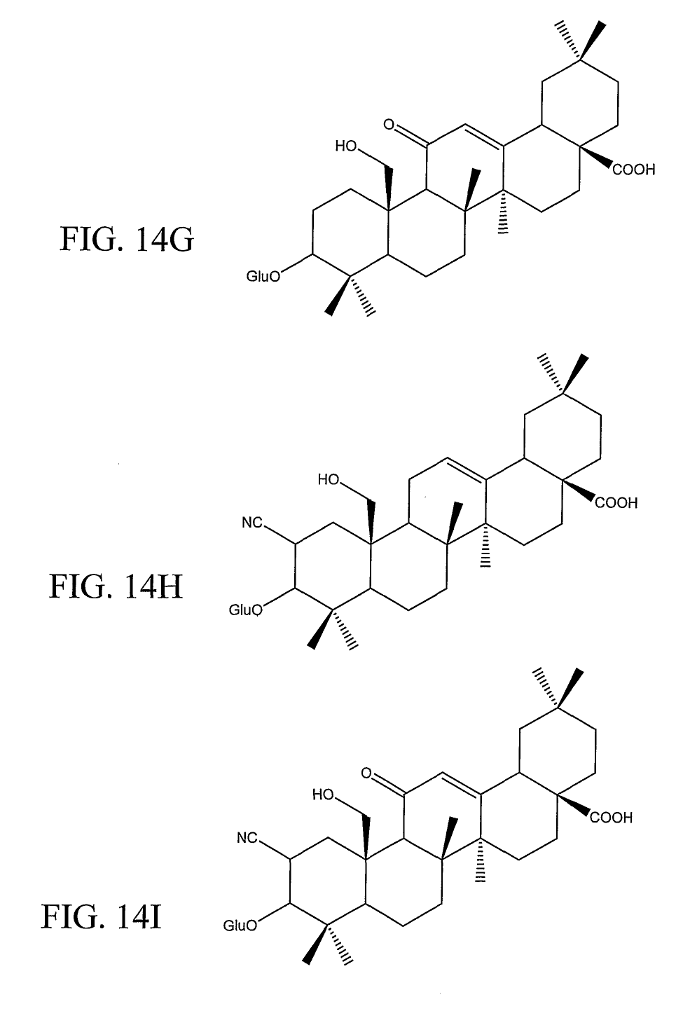

It will be appreciated that the AMR analogs of the invention can contain one or more asymmetrically substituted carbon atoms (i.e., carbon centers). The presence of one or more of the asymmetric centers in an analog of the invention, such as those shown in FIGS. 13 and 14A-14F, can give rise to stereoisomers, and in each case the invention is to be understood to extend to all such stereoisomers, including enantiomers and diastereomers, and mixtures including racemic mixtures thereof.

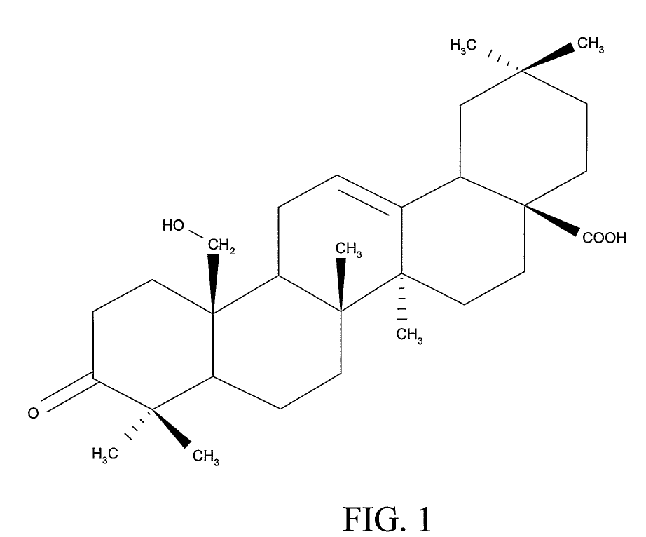

In one embodiment, the chemical structure (II) shown in FIG. 13 excludes the chemical structure of AMR (structure I; FIG. 1) or pharmaceutically acceptable salts thereof.

FIGS. 14A-14I show AMR analogs of the invention. In the analog of FIG. 14A (structure (III)), a —CO group is incorporated in the cyclohexane ring at the carbon alpha to the unsaturation, at position C11 of the molecule. In FIG. 14B, a —CN group is incorporated in the cyclohexane ring alpha to the keto group of C3, at position C2 of the molecule (structure (IV)). The AMR analog shown in FIG. 14C (structure (V)) has both the substituents (—CO and —CN) incorporated into the structures of FIGS. 14A and 14B. The AMR analogs shown in FIGS. 14D-14F (structures (VI), (VII), and (VIII)) have a substitution of the oxygen atom of the —CO group at C3 of amooranin with CH₃COO, resulting in the 3-O-acetyl derivative of structures (III)-(V) (FIGS. 14A-14C). The AMR analogs shown in FIGS. 14G-14I (structures (IX), (X), and (XI)) can be obtained by the replacement of the acetate group (CH₃COO—) at position C3 in structures (VI), (VII), and (VIII) of FIGS. 14D-14F by a glycosyl residue (glucose molecule).

The AMR compounds of the invention are useful for various non-therapeutic and therapeutic purposes. It is apparent from testing that the AMR compounds of the invention are effective for reducing aberrant cell growth. Because of the anti-proliferative properties of the compounds, they are useful to reduce unwanted cell growth in a wide variety of settings including in vitro and in vivo. They are also useful as standards and for teaching demonstrations. As disclosed herein, the AMR compounds are also useful prophylactically and therapeutically for treating cancer cells in animals and humans.

Therapeutic application of the AMR compounds and compositions containing them can be accomplished by any suitable therapeutic method and technique presently or prospectively known to those skilled in the art. Further, the AMR compounds of the invention have use as starting materials or intermediates for the preparation of other useful compounds and compositions.

AMR compounds of the invention may be locally administered at one or more anatomical sites, such as sites of unwanted cell growth. AMR compounds of the invention may be systemically administered, such as intravenously or orally, in combination with a pharmaceutically acceptable carrier such as an inert diluent, or an assimilable edible carrier for oral delivery. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active agent (i.e., AMR, or pharmaceutically acceptable salts or analogs of AMR) may also be administered intravenously or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the AMR compound (i.e., AMR, or a pharmaceutically acceptable salt or analog of AMR) in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the AMR compounds may be applied in pure-form, i.e., when they are liquids. However, it will generally be desirable to administer them topically to the skin as compositions, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the AMR compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver the AMR compound to the skin are disclosed in Jacquet et al (U.S. Pat. No. 4,608,392), Geria (U.S. Pat. No. 4,992,478), Smith et al. (U.S. Pat. No. 4,559,157) and Woltzman (U.S. Pat. No. 4,820,508).

Useful dosages of the pharmaceutical compositions of the present invention can be determined by comparing their in vitro activity, and in vivo activity in animal models. Methods for the extrapolation of effective dosages in mice, and other animals, to humans are known to the art; for example, see U.S. Pat. No. 4,938,949.

Accordingly, the present invention includes a pharmaceutical composition comprising an AMR compound (i.e., AMR, or pharmaceutically acceptable salt or analog thereof) in combination with a pharmaceutically acceptable carrier. Pharmaceutical compositions adapted for oral, topical or parenteral administration, comprising an amount of AMR compound constitute a preferred embodiment of the invention. The dose administered to a patient, particularly a human, in the context of the present invention should be sufficient to achieve a therapeutic response in the patient over a reasonable time frame. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

A suitable dose(s) is that amount that will reduce proliferation of the target cell(s). In the context of cancer, a suitable dose(s) is that which will result in a concentration of the active agent in cancer tissue, such as a malignant tumor, which is known to achieve the desired response. The preferred dosage is the amount which results in maximum inhibition of cancer cell growth, without unmanageable side effects. Administration of an AMR compound can be continuous or at distinct intervals, as can be determined by a person of ordinary skill in the art.

To provide for the administration of such dosages for the desired therapeutic treatment, new pharmaceutical compositions of the invention will advantageously comprise between about 0.1% and 45%, and especially, 1 and 15%, by weight of the total of one or more of the new compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

Mammalian species which benefit from the disclosed methods include, but are not limited to, primates, such as apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. Other species that may benefit from the disclosed methods include fish, amphibians, avians, and reptiles. As used herein, the terms “patient” and “subject” are used interchangeably and are intended to include such human and non-human species. Likewise, in vitro methods of the present invention can be carried out on cells of such human and non-human species.

Patients in need of treatment using the methods of the present invention can be identified using standard techniques known to those in the medical or veterinary professions, as appropriate.

As used herein, the terms “cancer” and “cancerous” refer to or describe the physiological condition in mammals that is typically characterized by unregulated cell growth. The cancer may be multi-drug resistant (MDR) or drug-sensitive. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

Other non-limiting examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas.

As used herein, the term “tumor” refers to all neoplastic cell growth and proliferation, whether malignant or benign, and all pre-cancerous and cancerous cells and tissues. For example, a particular cancer may be characterized by a solid mass tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue. However, some primary tumors are not palpable and can be detected only through medical imaging techniques such as X-rays (e.g., mammography), or by needle aspirations. The use of these latter techniques is more common in early detection. Molecular and phenotypic analysis of cancer cells within a tissue will usually confirm if the cancer is endogenous to the tissue or if the lesion is due to metastasis from another site.

According to the method of the subject invention, AMR, or a pharmaceutically acceptable salt or analog thereof, can be administered to a patient by itself, or co-administered with another agent. Co-administration can be carried out simultaneously (in the same or separate formulations) or consecutively. Furthermore, according to the method of the subject invention, AMR, or a pharmaceutically acceptable salt or analog thereof, can be administered to a patient as adjuvant therapy. For example, AMR, or a pharmaceutically acceptable salt or analog thereof, can be administered to a patient in conjunction with chemotherapy. In one embodiment, the AMR compound is co-administered to multi-drug resistant (MDR) cancer cells, in vitro or in vivo, with another anti-cancer agent, such as an anthracycline, such that the AMR compound makes the target MDR cells more sensitive to the co-administered anti-cancer agent, through the AMR compound's P-gp efflux blocking activity.

Thus, the AMR compounds of the subject invention (AMR, or a pharmaceutically acceptable salt or analog thereof), whether administered separately, or as a pharmaceutical composition, can include various other components as additives. Examples of acceptable components or adjuncts which can be employed in relevant circumstances include antioxidants, free radical scavenging agents, peptides, growth factors, antibiotics, bacteriostatic agents, immunosuppressives, anticoagulants, buffering agents, anti-inflammatory agents, anti-pyretics, time-release binders, anesthetics, steroids, and corticosteroids. Such components can provide additional therapeutic benefit, act to affect the therapeutic action of the AMR compound, or act towards preventing any potential side effects which may be posed as a result of administration of the AMR compound. The AMR compounds of the subject invention can be conjugated to a therapeutic agent, as well.

Additional agents that can be co-administered to target cells in vitro or in vivo, such as in a patient, in the same or as a separate formulation, include those that modify a given biological response, such as immunomodulators. For example, proteins such as tumor necrosis factor (TNF), interferon (such as alpha-interferon and beta-interferon), nerve growth factor (NGF), platelet derived growth factor (PDGF), and tissue plasminogen activator can be administered. Biological response modifiers, such as lymphokines, interleukins (such as interleukin-1 (IL-1), interleukin-2 (IL-2), and interleukin-6 (IL-6)), granulocyte macrophage colony stimulating factor (GM-CSF), granulocyte colony stimulating factor (G-CSF), or other growth factors can be administered. In one embodiment, the methods and compositions of the invention incorporate one or more agents selected from the group consisting of anti-cancer agents, cytotoxic agents, chemotherapeutic agents, and anti-signaling agents.

In another aspect, the subject invention pertains to the immunosuppressive use of the subject AMR compounds. The AMR compounds of the invention can be used to reduce, suppress, inhibit, or prevent unwanted immune responses. Thus, the AMR compounds of the subject invention are useful for treatments of humans or animals requiring immunosuppression. Examples of conditions for which immunosuppression is desired include, but are not limited to, treatment or prevention of autoimmune diseases such as diabetes, lupus, and rheumatoid arthritis. Immunosuppression is also frequently needed in conjunction with organ transplants. Immunosuppressive agents can also be utilized when a human or animal has been, or may be, exposed to superantigens or other factors known to cause overstimulation of the immune system. The AMR compounds of the subject invention are also useful as standards to assess the activity of other putative immunosuppressive agents.

A further aspect of the subject invention provides a process for obtaining isolated AMR from Amoora rohituka plant material. In one embodiment, the process includes (a) providing dried Amoora rohituka plant material in liquid or powder form; (b) performing an extraction on the liquid or powder with an extraction solvent to obtain an extract; (c) evaporating the extract to obtain a residue; (d) suspending the residue in a solvent; (e) performing additional extractions with petroleum ether and ethyl acetate; (f) evaporating the ethyl acetate fraction to obtain a residue; (g) dissolving the residue in ethyl acetate to obtain a solution; (h) precipitating the solution with an effective amount of solvent (such as a petroleum ether, methylene chloride, and ethyl acetate solvent system (such as 4:1:1)), and obtaining the filtrate; (i) eluting the concentrated filtrate with the same solvent system to obtain fractions; (j) subjecting the fractions to thin layer chromatography; (k) evaporating the solvents from the fraction having an Rf value of about 0.49 to obtain a residue; (l) crystallizing the residue; (m) washing and drying the crystals; and (n) recrystallizing to obtain a pure solid. In one embodiment, the Rf value of the fraction is 0.42-0.55. In another embodiment, the Rf value of the fraction is 0.45-0.53. In another embodiment, the Rf value of the fraction is 0.47-0.51. In another embodiment, the Rf value of the crystals is 0.48-0.50.

In a preferred embodiment, dried bark of the Amoora rohituka tree is powdered into coarse form and is extracted with 200% proof ethanol in a Soxhlet extractor for 48 hours continuously. The extract is evaporated in a rotary evaporator at 50° C. and the residue is suspended in distilled water and extracted successively with petroleum ether and ethyl acetate. The ethyl acetate fraction is evaporated under vacuum at 40° C., the residue can be dissolved in ethyl acetate, precipitated with an effective amount of a solvent (such as a solvent system containing petroleum ether, methylene chloride and ethyl actetate (such as 4:1:1)), and filtered. The filtrate is preferably concentrated by evaporation and subsequently taken on a silica gel column and eluted with the same solvent system. The fractions having an Rf value of about 0.49 (on thin layer chromatography (TLC)) are collected, evaporating the solvents at 40° C. under vacuum, followed by crystallization in methanol. In one embodiment, the Rf value of the fraction is 0.42-0.55. In another embodiment, the Rf value of the fraction is 0.45-0.53. In another embodiment, the Rf value of the fraction is 0.47-0.51. In another embodiment, the Rf value of the crystals is 0.48-0.50. The crystals are washed with cold methanol several times until they become colorless. The crystals can be dried and re-crystallized in methanol to obtain pure white fine solid (yield≦0.01%). A scientist skilled in the art of natural products purification can easily adapt the foregoing methods and substitute a variety of solvents and stationary phases for those described in the preferred embodiment of the invention. For example, solid-liquid extraction or liquid-liquid extraction may be used to obtain the isolated compound. In addition to chromatography, methods such as crystallization and partitioning can also be used to purify the desired compounds. See, for example, Brown G. G., Unit Operations, John Wiley & Sons, 1956; McCabe, W. L. and J. C. Smith, Unit Operations of Chemical Engineering, McGraw-hill, 1956; and Perry, R. H., and D. Green, Perry's Chemical Engineers' Handbook, 7^{th} Edition, McGraw-Hill, 1997, which are incorporated herein by reference in their entirety.

There is no particular limitation as to the method for extracting AMR from the plant. For example, extraction with various solvents or supercritical fluid extraction is applicable. There is no particular limitation as to the solvents used for extraction of AMR from the plant. Examples of suitable solvents include aqueous media such as water, inorganic salt aqueous solution and buffer solutions, and organic solvents such as alcohol, hexane, toluene, petroleum ether, benzene, ethyl acetate, chloroform, dichloromethane, 1,1,2-trichloroethane, di-methylsulfoxide, and acetone, among which alcohol is preferred. Water can be water, distilled water, deionized water, or pure water. Examples of buffer solution that may be used include phosphate buffer and citrate buffer.

Examples of the alcohol that may be used include monohydric alcohols such as methanol, ethanol, propanol and butanol, and multi-hydric alcohols such as propylene glycol and glycerol, among which a monohydric alcohol is preferred, and particularly ethanol is preferred. These solvents may be used alone or as a mixture. As the mixed solvent, water-containing alcohols are preferred. Water-containing monovalent alcohols are more preferred, and water-containing ethanol is particularly preferred.

In extracting AMR from the plant material, it is appropriate to use a solvent which is suitable for therapeutics, such as water, water-containing ethanol, or anhydrous ethanol.

For extraction, the solvent may be used, for example, in an amount of 0.1 to 10,000 parts by weight preferably 1 to 100 parts by weight based on 1 part by weight of the plant. There is no particular limitation as to the extraction temperature, but the extraction is preferably carried out at 0 to 100° C., more preferably at 20 to 90° C. There is no particular limitation as to the time for extraction, but it may preferably be conducted, for example, for a period of 1 minute to 1 week, more preferably 30 minutes to 1 day.

There is no particular limitation as to the apparatus used for extraction, and a vessel designed for efficient extraction, a stirrer, a reflux condenser, a Soxhlet extractor, a homogenizer, a shaker, a supersonic generator, etc., may be used. The liquid extract may be treated by means of various solid-liquid separation such as sedimentation, cake filtration, clear filtration, centrifugal filtration, centrifugal sedimentation, compression separation or filter press.

For preparing the plant powder, all or parts of the plant containing AMR may be used, such as leaves, branches, stems, stem bark, roots, seeds, cultured cells or organs, or callus. The stem bark is preferred. The plant material may be used as such or after being treated physically or chemically or biologically. Examples of the method of physical or chemical treatment are drying, freeze-drying, disruption, and extraction. The physically or chemically treated matter includes dried matter, freeze-dried matter, disrupted matter and extracted matter. The extracted matter includes the residue of the plant obtained after the extraction.

For preparing the plant powder, the preferably dried Amoora rohituka plant material can be crushed with a compression crusher, such as jaw crusher, gyratory crusher or cone crusher; shearing machine, such as cutter mill or shredder; impact crusher, such as hammer crusher; roll mill, such as roll crusher; rotary mill, such as disintegrator or cage mill; screw mill, such as coffee mill; rolling mill, such as edge runner; hammering mill, such as stamp mill; roller mill, such as centrifugal roller mill, ball bearing mill, bowl mill, or zego mill; high speed rotary mill, such as swing hammer mill, pin mill, cage mill, turbo-type mill, or centrifugal mill; vessel vibrating mill, such as rolling ball mill, vibrating ball mill, planetary ball mill, or CF mill; jet mill, such as flow-pipe type mill, stirring tank mill, annular-type mill, air suction type mill, impact plate impact miller, or fluidized bed mill; crusher, such as ultrasonic shredder; stone mortar or mortar. The product obtained by the aforementioned method may further be processed physically or chemically to give plant powder.

The plant powder can be coarse or fine. The average particle size of the plant powder is preferably 0.1 μm to 1 mm, more preferably 1 to 100 μm, and particularly 2 to 50 μm in a dry state. The average particle size of the plant powder in a dry state can be determined, for example, by a laser diffraction particle distribution analyzer. Alternatively, when the plant powder is swelled with a 1:1 mixture of glycerol and water, the average particle size of the powder is preferably 1 μm to 10 mm, more preferably 10 μm to 1 mm, and particularly 20 to 500 μm. The average particle size of the plant powder in a swelling state can be determined, for example by observation with a microscope.

Methods of drying are known in the art. For example, the method of drying can involve drying under heating and reduced pressure, drying under heating and atmospheric pressure, or drying with a spray drier or with drum drier, or freeze-drying, among which drying under heating and reduced pressure or freeze-drying is preferred.

As used herein, the terms “treat” or “treatment” refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. For example, treatment with an AMR compound may include reduction of undesirable cell proliferation, reduction of multidrug resistance (MDR), and/or induction of apoptosis and cytotoxicity. Reduction in cellular proliferation can occur through one or more of G₂/M cell cycle arrest, caspase activation, and apoptosis. “Treatment” can also mean prolonging survival as compared to expected survival if not receiving treatment. Those in need of treatment include those already with the condition or disorder as well as those prone to have the condition or disorder or those in which the condition or disorder is to be prevented.

As used herein, the term “(therapeutically) effective amount” refers to an amount of the AMR compound or other agent (e.g., a drug) effective to treat a disease or disorder in a mammal. In the case of cancer, the therapeutically effective amount of the agent may reduce (i.e., slow to some extent and preferably stop) unwanted cellular proliferation; reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve, to some extent, one or more of the symptoms associated with the cancer. To the extent the AMR compound prevents growth and/or kill existing cancer cells, it may be cytostatic and/or cytotoxic. For cancer therapy, efficacy can, for example, be measured by assessing the time to disease progression (TTP) and/or determining the response rate (RR).

As used herein, the term “growth inhibitory amount” of the AMR compound refers to an amount which inhibits growth or proliferation of a target cell, such as a tumor cell, either in vitro or in vivo, irrespective of the mechanism by which cell growth is inhibited. In a preferred embodiment, the growth inhibitory amount inhibits (i.e., slows to some extent and preferably stops) or proliferation growth of the target cell in vivo or in cell culture by greater than about 20%, preferably greater than about 50%, most preferably greater than about 75% (e.g., from about 75% to about 100%).

The terms “cell” and “cells” are used interchangeably herein and are intended to include either a single cell or a plurality of cells, in vitro or in vivo, unless otherwise specified.

As used herein, the term “anti-cancer agent” refers to a substance or treatment that inhibits the function of cancer cells, inhibits their formation, and/or causes their destruction in vitro or in vivo. Examples include, but are not limited to, cytotoxic agents (e.g., 5-fluorouracil, TAXOL), chemotherapeutic agents, and anti-signaling agents (e.g., the PI3K inhibitor LY).

As used herein, the term “cytotoxic agent” refers to a substance that inhibits or prevents the function of cells and/or causes destruction of cells in vitro and/or in vivo. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³², and radioactive isotopes of Lu), chemotherapeutic agents, toxins such as small molecule toxins or enzymatically active toxins of bacterial, fungal, plant or animal origin, and antibodies, including fragments and/or variants thereof.

As used herein, the term “chemotherapeutic agent” is a chemical compound useful in the treatment of cancer, such as, for example, taxanes, e.g., paclitaxel (TAXOL, BRISTOL-MYERS SQUIBB Oncology, Princeton, N.J.) and doxetaxel (TAXOTERE, Rhone-Poulenc Rorer, Antony, France), chlorambucil, vincristine, vinblastine, anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON, GTx, Memphis, Tenn.), and anti-androgens such as flutamide, nilutamide, bicalutamide, leuprolide, and goserelin, etc. In a preferred embodiment, the chemotherapeutic agent is one or more anthracyclines. Anthracyclines are a family of chemotherapy drugs that are also antibiotics. The anthracyclines act to prevent cell division by disrupting the structure of the DNA and terminate its function by: (1) intercalating into the base pairs in the DNA minor grooves; and (2) causing free radical damage of the ribose in the DNA. The anthracyclines are frequently used in leukemia therapy. Examples of anthracyclines include daunorubicin (CERUBIDINE), doxorubicin (ADRIAMYCIN, RUBEX), epirubicin (ELLENCE, PHARMORUBICIN), and idarubicin (IDAMYCIN).

As used herein, the term “AMR compound” is intended to refer to AMR (25-hydroxy-3-oxoolean-12-en-28-oic acid, as shown in FIG. 1), or a pharmaceutically acceptable salt or analog of AMR. As used herein, the term “isolated” with respect to AMR or an AMR compound refers to the compound substantially free from the medium in which it naturally occurs, e.g., from Amoora rohituka plant material or plant extract. However, an isolated AMR compound may also be obtained by appropriate chemical synthesis reactions known to those skilled in the art (Greene, T. W. and Wuts, P. G. M. “Protective Groups in Organic Synthesis” John Wiley & Sons, Inc. New York. 3rd Ed. pg. 819, 1999; Honda, T. et al. Bioorg. Med. Chem. Lett., 1997, 7:1623-1628; Honda, T. et al. Bioorg. Med. Chem. Lett., 1998, 8:2711-2714; Konoike, T. et al. J. Org. Chem., 1997, 62:960-966; Honda, T. et al. J. Med. Chem., 2000, 43:4233-4246).

Following are examples that illustrate materials, methods, and procedures for practicing the invention. The examples are illustrative and should not be construed as limiting. All percentages disclosed herein, whether supra or infra, are by weight and all solvent proportions are by volume unless otherwise noted.

### Materials and Methods

Isolation of Amooranin (AMR). The dried bark of Amoora rohituka tree was powdered into coarse form and extracted with 200% proof ethanol in a Soxhlet extractor for 48 hours continuously. The extract was evaporated in a rotary evaporator at 50° C. and the residue was suspended in distilled water and extracted successively with petroleum ether and ethyl acetate. The ethyl acetate fraction was evaporated under vacuum at 40° C. and the residue was dissolved in a minimum quantity of ethyl acetate and precipitated with the solvent system containing petroleum ether, methylene chloride and ethyl acetate (4:1:1) and filtered. The filtrate was concentrated by evaporation and passed through a silica gel column and eluted with the same solvent system. The fractions having Rf value of about 0.49 (on TLC) were collected and crystallized in methanol, after evaporating the solvents at 40° C. under vacuum. The crystals were washed with cold methanol until they became colorless. The crystals were then dried and re-crystallized in a minimum quantity of methanol to obtain a pure white fine solid (yield≦0.01%). The isolated compound was highly pure when analyzed by thin layer chromatography and it showed significant cytotoxicity against tumor cells. The other advantage of this compound is its good solubility in pharmacologically accepted solvent systems.

Cells and cell culture. Human leukemia cell lines (CEM), and its vinblastine-resistant subline, CEM/VLB were obtained from Dr. William Beck (University of Illinois). Human colon carcinoma (SW620) and its MDR cell line, SW620/Ad-300 cell lines were obtained from Dr. S. Bates (National Cancer Institute). These cell lines were maintained in RPMI medium supplemented with 10% fetal bovine serum, 100 IU/μl of penicillin and 100 μg/ml of streptomycin in a humidified atmosphere of 5% CO₂ at 37° C. The resistant cells were occasionally (once in two months) challenged in medium containing 0.1 μM of vinblastine for CEM/VLB cells and 300 ng/ml of DOX for SW 620/Ad-300 cells to maintain their resistance level.

Human breast adenocarcinoma MCF-7 cells, its multidrug resistant subclone MCF-7/TH and mammary epithelial MCF-10A cells were cultured in Dulbecco's Modified Essential Medium (LIFE TECHNOLOGIES, INC., MD) containing 10% Fetal Bovine Serum and antibiotics (100 units/ml penicillin, 100 μg/ml streptomycin sulfate), in a 5% CO₂ incubator at 37° C.

Chemicals and reagents. 3-(4,5-Dimethylthiozal-2-yl)-2,5 diphenyl tetrazolium bromoide (MTT) survival assay kit and in situ cell death detection kit were purchased from ROCHE Molecular Biochemicals, Indianapolis (IN). Fluorescein Caspase (VAD) Activity Kit and Caspase-8 (LETD) Activity Kit were purchased from INTERGEN Company (NY).

Drugs. Doxorubicin hydrochloride (DOX) was purchased from SIGMA Chemical Co., MO. Amooranin (AMR) was isolated as described above. The chemical structure of amooranin is shown in FIG. 1.

Cytotoxicity Assay (Examples 1-5). Drug sensitive and resistant tumor cells were treated with AMR, DOX or their combination for analysis of cytotoxicity to individual drugs as well as their combination (Rabi, T. et al. Phytother Res, 2001, 15:1-3; Rabi, T. et al. “Amooranin overcomes cellular drug resistance and acts synergistically with doxorubicin in multidrug resistant human colon carcinoma and leukemia cell lines” Proc. 2001, AACR-NCI-EORTC International Conference: Molecular Targets and Cancer Therapeutics: Discover, Biology and Clinical Applications, 2001, p. 88). Cell proliferation kit I (MTT) from Roche Diagnostics GmbH (Indianapolis, Ind.) was used for cytotoxicity assays and manufacturers protocol was followed for the assay. Dose modification factor (DMF) was calculated by dividing the DOX IC₅₀ value in DOX+AMR combination by the IC₅₀ value for DOX alone treatment.

DNA Histogram Analysis. Following drug exposure for 48 hours, cells were centrifuged and resuspended in propidium iodide-hypotonic citrate solution for 1 hour before flow cytometric analysis of cell cycle distribution (Ramachandran, C. et al. Biochem Pharmacol, 1995, 49:545-552).

Cellular DOX Content. Log-phase cells (1×10⁶) were incubated with 2 μg DOX in the absence or presence of AMR (1 or 2 μg/ml) for 1 hour at 37° C. Cellular DOX fluorescence was analyzed in a Coulter Elite flow cytometer. Fluorescence emission (above 530 nm) from at least 10,000 cells were collected, amplified and scaled to generate a single parameter histogram (Ramachandran, C. et al. Biochem Phammacol, 1995, 49:545-552).

P-gp Expression. Flow cytometric analysis of P-gp expression was performed using MRK16 monoclonal antibody (Mab; KAMIYA Biochemical Co., Seattle, Wash.) according to our published procedure (Ramachandran, C. et al Biochem Pharmacol, 1998, 56:709-718).

Photolabeling of Plasma Membrane. Plasma membranes from tumor cells were prepared according to our published procedure (Ramachandran, C. et al Biochem Pharmacol, 1998, 56:709-718; Chen, G. et al. Cancer Res, 1993, 53:2544-2547). Protein content of the membrane preparation was determined by the Bradford assay. The membrane vesicles (50 μg protein) were photolabeled in 40 mM potassium phosphate buffer containing 10 μM CaCl₂, 4% dimethyl sulfoxide, and 0.2 μM [³H]-azidopine (10 μCi) in a final volume of 50 μl. This mixture was pre-incubated for 1 hour at room temperature in the dark in the absence or presence of 1, 2, 4 μg/ml AMR or 2 μg/ml DOX+4 μg/ml AMR. The photolabeling mixture was then irradiated in a UV crosslinker for 20 minutes. Photolabeled membrane protein preparations were separated by 7.5% sodium dodecyl sulfate-polyacrylamide gel electrophoresis. Covalent incorporation of [³H]-azidopine was detected by fluorography using Amplify (AMERSHAM PHARMACIA, Piscataway, N.J.).

Cytotoxicity assay (Examples 6-8). MTT assay was used to determine drug sensitivity (Hansen, M. B. et al. J Immunol Methods, 1989, 119:203-210). Cells (1×10⁴) were grown in 96-well plates and after 48 hours, the medium was replaced with new medium. The cells were incubated at 37° C. with varying concentrations of AMR for 48 hours at 37° C. Cell viability was then assayed by adding MTT dye (500 μg/ml). Six hours after incubation period, MTT containing medium was removed. The formazan crystals were dissolved in 100 μl/well dimethyl sulfoxide, and the plates were read in BIO-RAD Benchmark Microplate Reader at 570 nm wavelength. All experiments were repeated three times with three replications in each and the mean values were estimated. Student's t test was used to determine the significant difference between IC₅₀ values.

Cell cycle analysis. The number of cells in each stage of the cell cycle was monitored by flow cytometric analysis of cellular DNA content after staining with 1 mg/ml propidium iodide (Chen, G. et al. Cancer Res, 1993, 53:2544-2547). Data was analyzed to determine the percentage of cells at each phase of the cell cycle (G₀+G₁, S, and G₂+M).

Analysis of DNA fragmentation by agarose gel electrophoresis. To assess the pattern of DNA cleavage caused by AMR, agarose gel electrophoresis was performed as described by Cohen and Duke (Cohen, J. J. and Duke, R. C. J Immunol, 1984, 132:38-42). Briefly, control and drug-treated cells (2×10⁶ cells) were lysed with 0.5 ml lysis buffer containing 0.2% Triton X-100 at room temperature for 30 minutes. The supernatant fractions were collected by centrifugation at 12,000×g for 30 minutes. The DNA in these fractions was precipitated overnight with 100 μl 5 M sodium chloride and 0.5 ml 2-isopropanol at −20° C. The DNA was dissolved in 20 μl of 10 mM Tris-HCl (pH 7.4) and 1 mM EDTA buffer. RNase (10 units) was added to the samples before incubating at 60° C. for 1 hour. An equal volume of loading buffer was added afterwards and the samples were subjected to electrophoresis on a 2% agarose gel in Tris borate buffer at 50 V for 3 hours. The agarose gel was stained with ethidium bromide, and DNA fragmentation pattern was visualized in a UV transilluminator. AMR-induced DNA fragmentation was also analyzed by TUNEL assay using in situ cell detection kit according to manufacturer's instructions. Briefly, cells were permeabilized in a solution containing 0.1% Triton X-100 and 0.1% (w/v) sodium citrate for 2 minutes. After washing in PBS, cells were incubated with label solution and terminal transferase. Cells were incubated without terminal deoxynucleotidyl transferase was used as negative control. DNase (1 μg/ml) treated cells incubated with label solution in the presence of terminal deoxynucleotidyl transferase (TUNEL reaction mixture) was used as positive control and the details of TUNEL assay have been described earlier (Ramachandran, C. et al. Anticancer Res, 1997, 17:3369-3376).

Detection of apoptosis and caspase activity. Breast carcinoma or breast epithelial cells were incubated for 48 hours in the presence or absence of AMR (1-8 μg/ml). AMR-induced apoptosis was evaluated by Fluorescein Caspase (VAD) Activity Kit and caspase-8 (LETD) Activity Kit according to manufacturer's instructions (Carcia-Calvo, M. et al. J Biol Chem, 1998, 273:32608-32613).

Xenograft studies with AMR in nude mice. Male athymic nude (nu/nu) mice of the BALB/c strain (4-5 weeks old) were purchased from Taconic Labs, NY. SW620 colon carcinoma cells (10⁶/site) in 0.1 ml of Hank's balanced salt solution were injected subcutaneously into the right flank of the nude mice using a 21 gauge needle. When the tumors reached approximately 0.5 cm³ size in volume (usually 14 days), chemotherapy with AMR was started involving six animals in each group with almost similar size tumors. The treatment groups consisted of 2 mg/kg doxorubicin, 0, 10, and 20 mg AMR/kg body weight intraperitoneal injections every week for three weeks. AMR was dissolved in 1:1 mixture of Cremophor EL and ethanol for administration and the control treatments were doxorubicin dissolved in phosphate dissolved saline and saline alone (0 mg/kg). Tumor size (length and width) was recorded on every fifth day of treatment along with body weight and survival data. Tumor volume was calculated using the following formula:

$$Tumor volume ( mm 3 ) = \frac{length ( mm ) \times width {( mm )}^{2}}{2}$$

Tumor growth rate was calculated as mean V₁/V₀ where V₁ is the volume on the evaluation day of treatment effect and V₀ is that on the first day of treatment. The growth rate index and therapeutic index estimates were calculated using the following formulae:

$$Growth Rate Index = \frac{\begin{matrix}Tumor growth rate \\ of a treated mouse\end{matrix}}{\begin{matrix}Average tumor growth \\ rate of control mouse\end{matrix}} \times 100$$

$$Therapeutic Ratio = 100 - Tumor Growth Rate Index ( GRI )$$

### Example 1

### Reversal of DOX Resistance by AMR

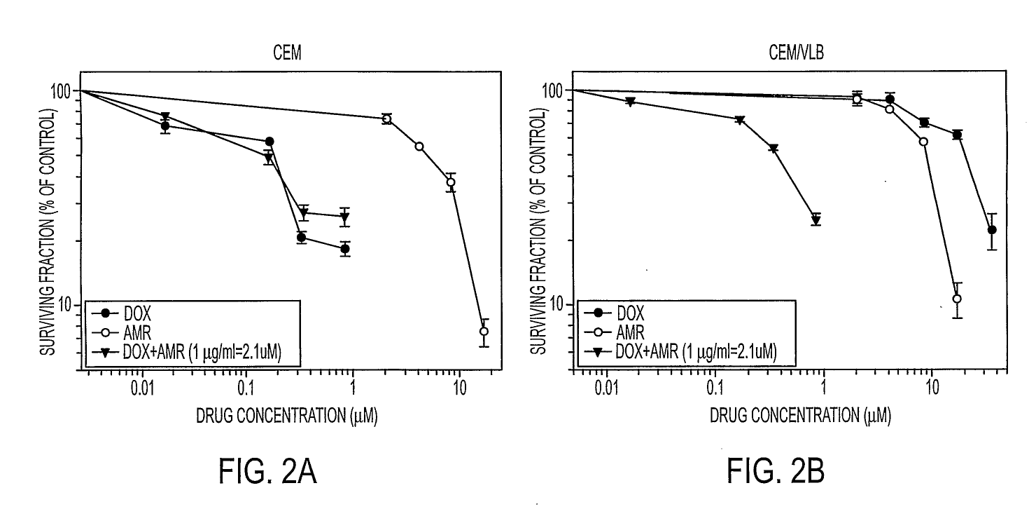

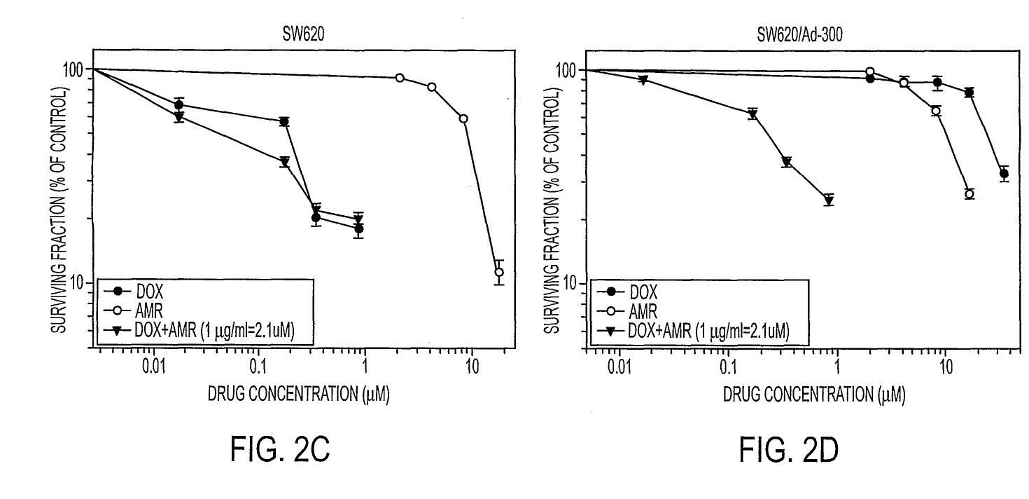

Sensitivity of human leukemia (CEM and CEM/VLB) and colon carcinoma (SW620 and SW620/AD-300) cells to AMR, DOX and their combination are presented in the dose response curves (FIG. 2). The IC₅₀ values of DOX, AMR and the combination are given in Table 1. The DOX IC₅₀ values (48 hours exposure) for sensitive human leukemia (CEM) and multidrug resistant CEM/VLB cell lines were 0.12 μg/ml and 13.4 μg/ml, respectively, indicating approximately 112-fold resistance in CEM/VLB. Cytotoxicity data (Table 1) showed that CEM/VLB cells were also 1.9-fold resistant to AMR than CEM cells. When CEM/VLB cells were co-incubated with varying concentrations of DOX and 1 μg/ml AMR, DOX IC₅₀ values was decreased from 13.4 to 0.25 μg/ml with a dose modification factor (DMF) of 53.6. However, CEM/VLB cells treated with DOX and AMR (1 μg/ml) combination had about 2-fold residual DOX resistance compared with parental CEM cells. The case was also similar in colon carcinoma cell lines. Based on IC₅₀ values, SW620/Ad-300 cells were approximately 125-fold resistant to DOX than sensitive SW620 parental cells. SW620/Ad-300 cells were also 1.6-fold resistant to AMR as compared to SW620 cells. When the resistant SW620/Ad-300 cells were co-incubated with AMR and DOX, DOX resistance was reversed significantly with a DMF of 108.

TABLE 1
Amooranin and doxorubicin IC50 values of human
leukemic and colon carcinoma cell lines

| | | | DOX IC₅₀ (μM) |
|---|---|---|---|
| | | | in DOX + AMR |
| | AMR IC₅₀ | DOX IC₅₀ | (1 μg/ml = 2.1 μM) |
| Cell Line | (μM) | (μM) | combination |
| Leukemia | | | |
| CEM | 5.31 ± 0.02 | 0.23 ± 0.02 | 0.15 ± 0.01 |
| CEM/VLB | 10.19 ± 0.11 | 23.10 ± 0.78 | 0.47 ± 0.04 |
| | | | (DMF = 50.9) |
| Colon carcinoma | | | |
| SW620 | 7.70 ± 0.15 | 0.26 ± 0.04 | 0.09 ± 0.01 |
| SW620/Ad-300 | 12.70 ± 0.27 | 28.88 ± 0.95 | 0.29 ± 0.04 |
| | | | (DMF = 99.6) |

Values are mean ± SD (n = 3)

### Example 2

### Cell Cycle Effects

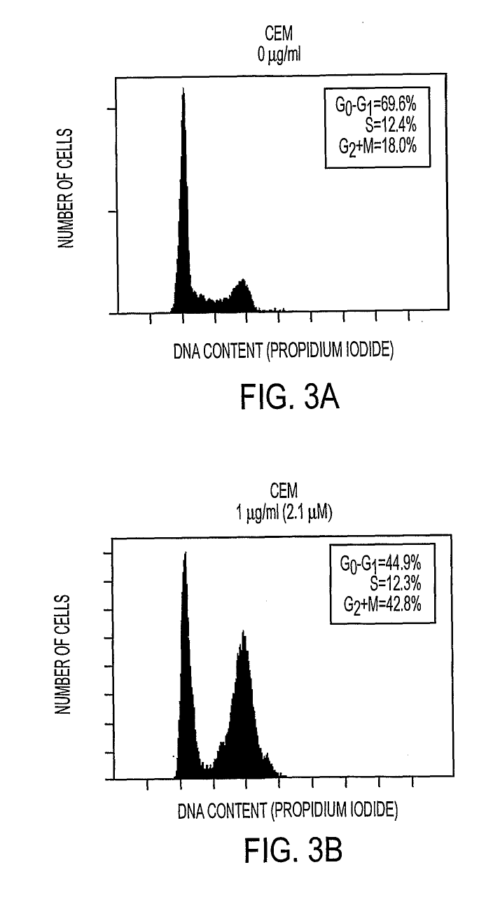

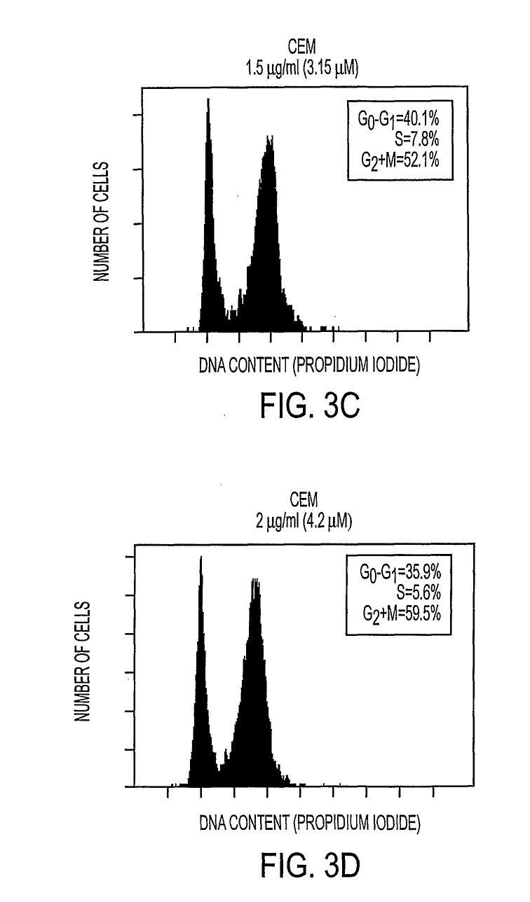

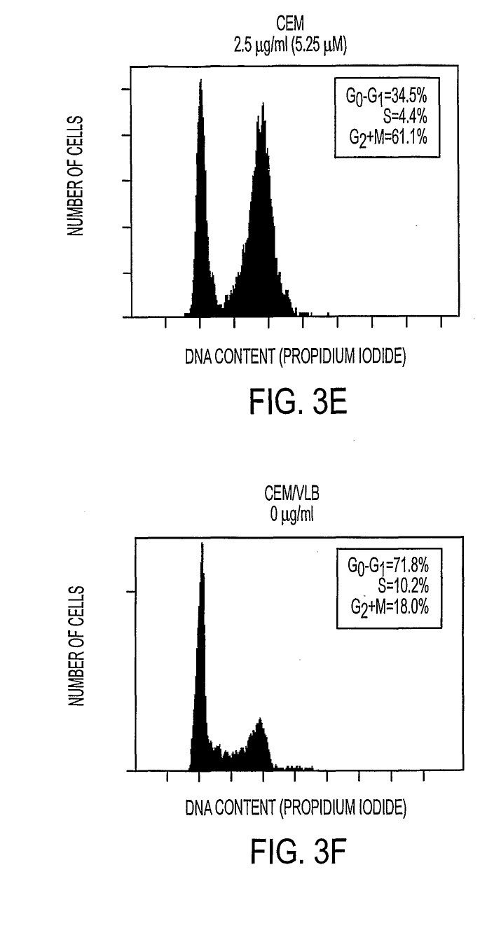

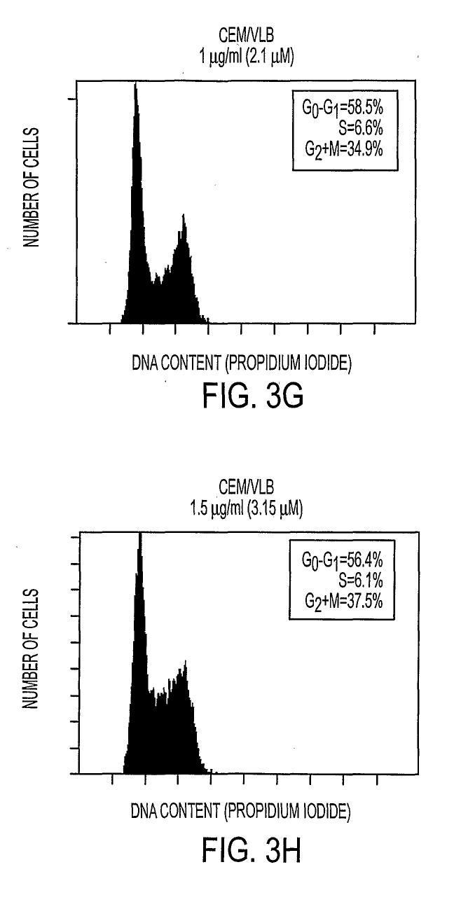

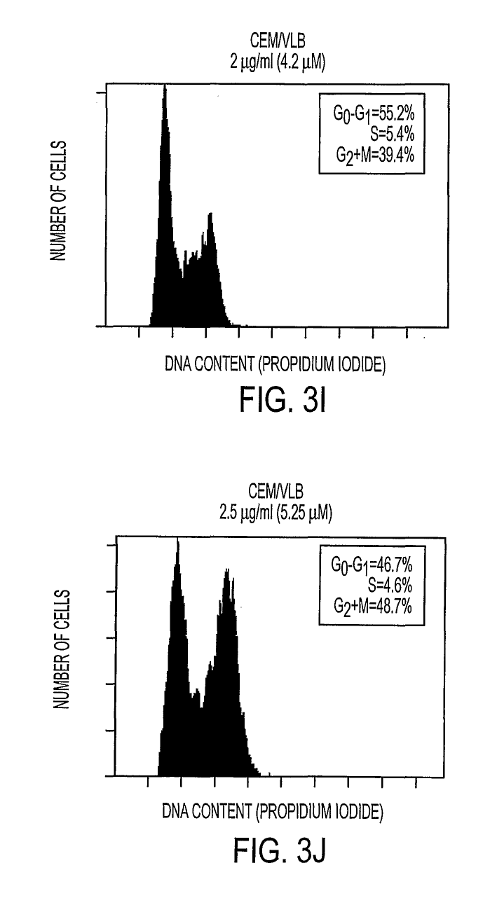

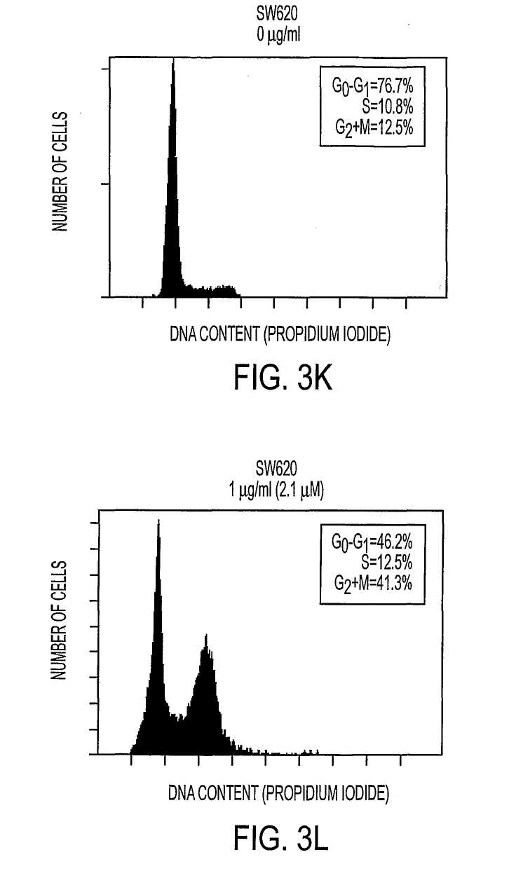

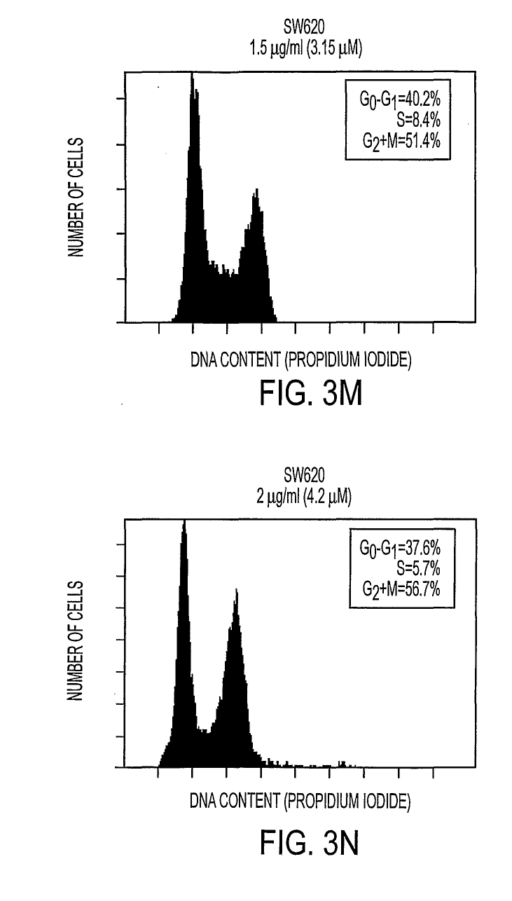

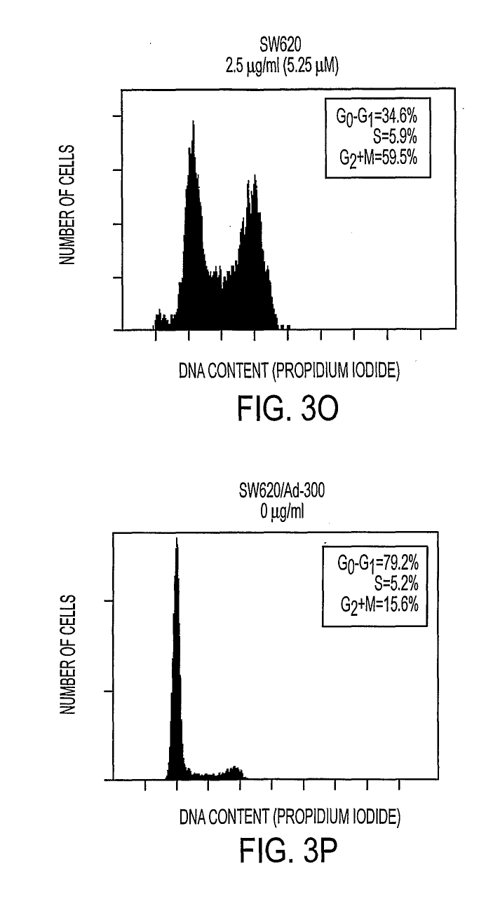

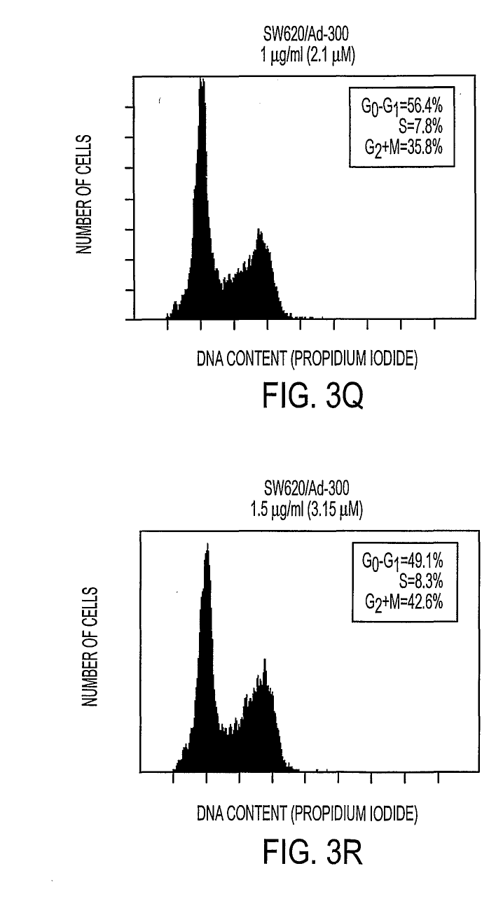

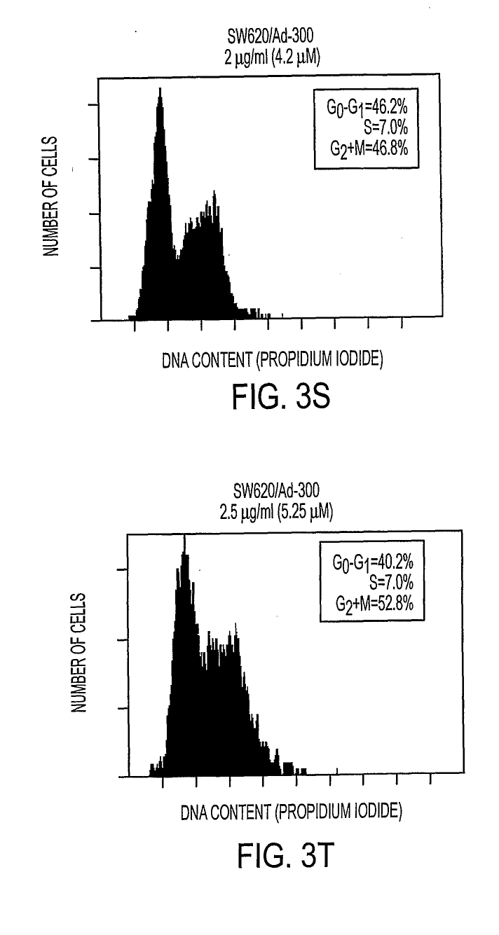

Following AMR treatment of tumor cells in vitro for 48 hours, cellular DNA content was analyzed by flow cytometry. DNA histograms in FIG. 3 are of cells exposed to 0-2.5 μg/ml AMR for 48 hours. In all four cell lines, AMR treatment caused an increase in the percentage of cells in G₂+M phase and a simultaneous decrease in G₀-G₁ population in a dose-dependent manner. This AMR-induced G₂+M arrest may be a prelude to their entry into apoptosis. The percentage of G₂+M phase cells was proportionately higher in drug sensitive CEM and SW620 cell lines compared with drug resistant CEM/VLB and SW 620/Ad-300 cell lines at every AMR concentrations. In human leukemia cell lines AMR treatment caused an increase in the percentage of G₂+M cells from 18.0% to 61.1% in CEM and from 18.0% to 48.7% in CEM/VLB cell lines. Similar cell cycle distribution was also evident in colon carcinoma cell lines. In SW 620 cell line, G₂+M percentage ranged from 12.5% to 59.5% as the AMR concentration increased from 0-2.5 μg/ml. In the multidrug resistant SW620/Ad-300 cell line G₂+M percentage increased from 15.6% to 52.8% with AMR treatment.

### Example 3

### Effect of AMR on Cellular DOX Accumulation

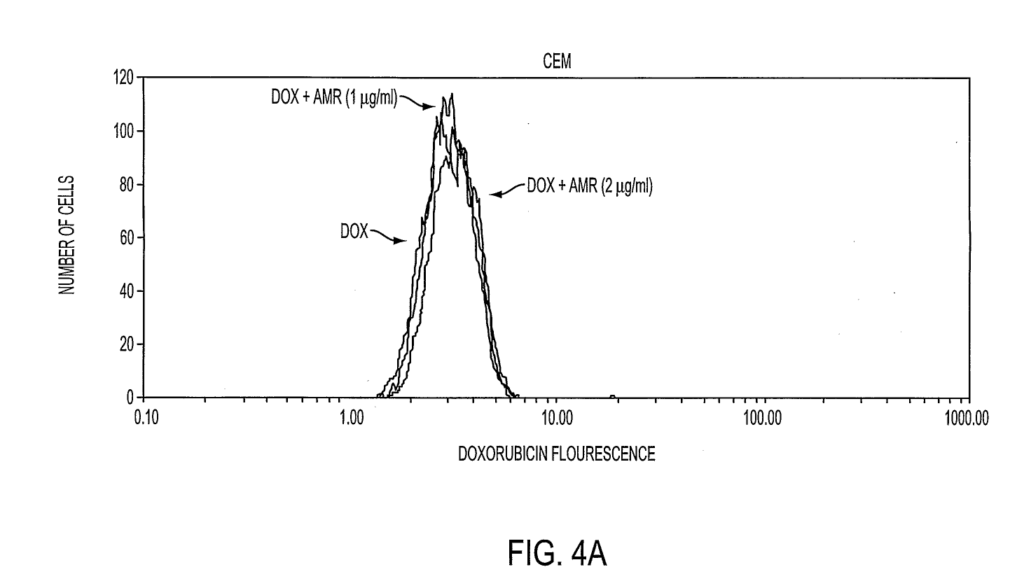

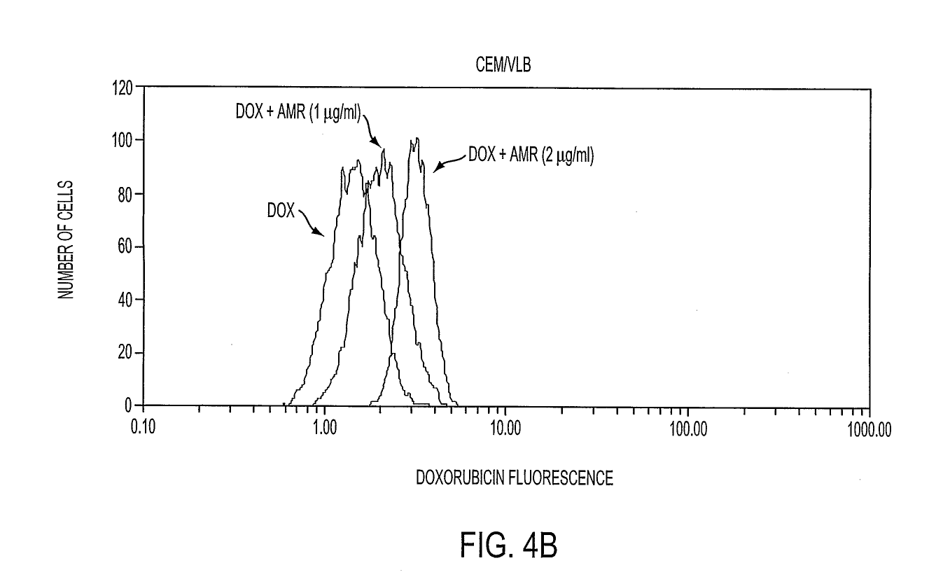

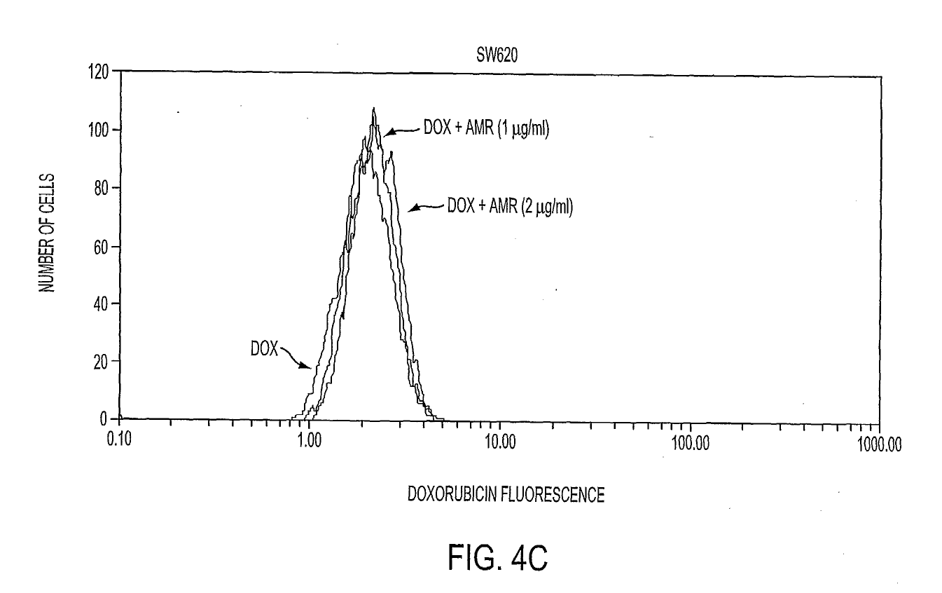

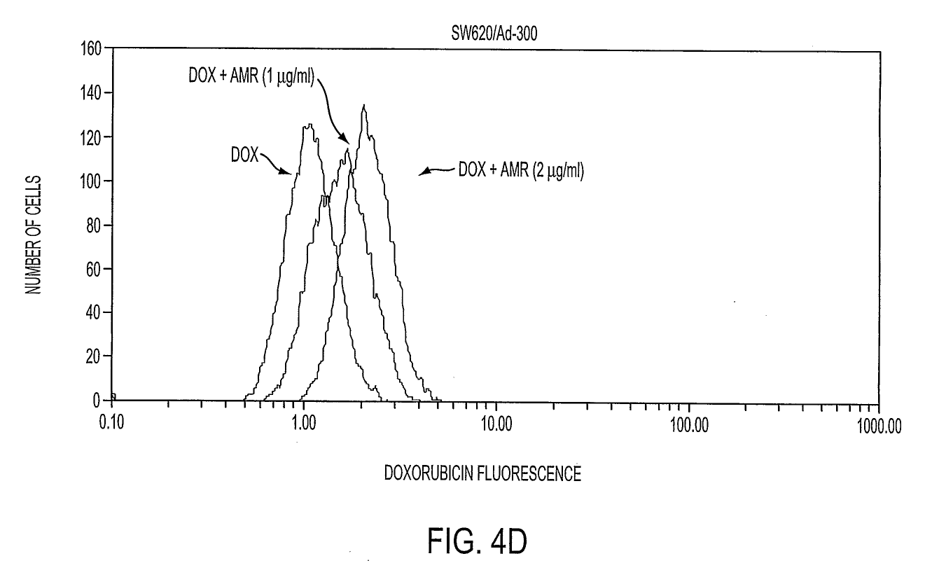

FIGS. 4A-4D shows cellular DOX accumulation in human leukemia and colon carcinoma cells incubated in the presence or absence of AMR. No significant change in the cellular DOX fluorescence was evident in the presence of 1 or 2 μg/ml AMR in sensitive CEM and SW620 cell lines. However, AMR at 1 and 2 μg/ml increased cellular DOX accumulation in a dose-dependent manner in multidrug resistant CEM/VLB and SW 620/Ad-300 cell lines.

### Example 4

### P-gp in MDR Cells

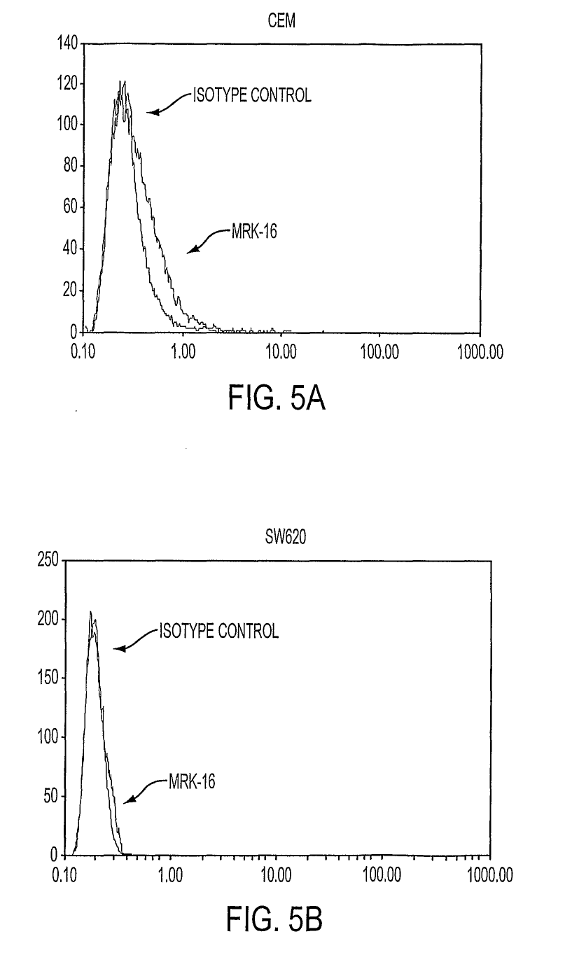

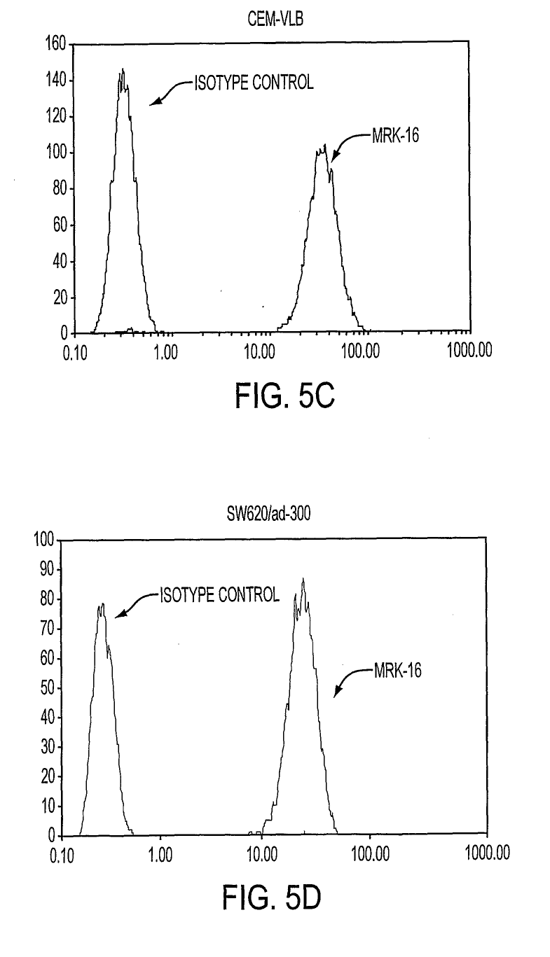

Drug sensitive human leukemia (CEM) and colon carcinoma (SW620) cells do not express P-gp (FIGS. 5A-5D), when cells were stained with MRK16 Mab and analyzed by flow cytometry. On the other hand, multidrug resistant cells such as CEM/VLB and SW620/Ad-300 express P-gp abundantly.

### Example 5

### Inhibition of [³H]-Azidopine Photolabeling of P-gp

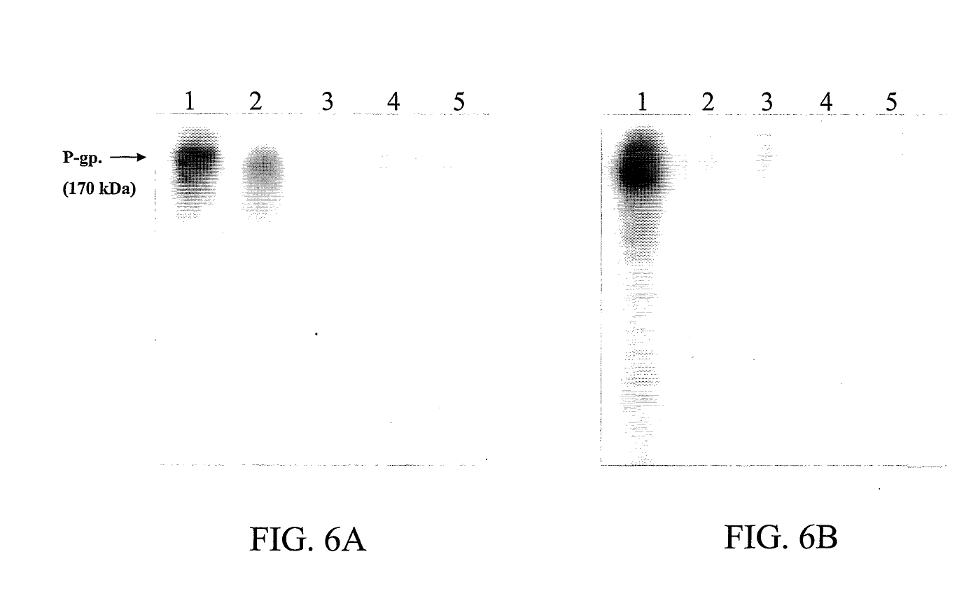

The inhibitory effect of AMR on photolabeling of P-gp with [³H]-azidopine in multidrug resistant (CEM/VLB and SW620/Ad-300) cells is shown in FIGS. 6A-6B. Multidrug resistant CEM/VLB and SW620/Ad-300 cells express abundant level of P-gp, that was labeled with [³H]-azidopine (lane 1 of FIGS. 6A and 6B). AMR at 1, 2, and 4 μg/ml inhibited azidopine binding of P-gp in a dose-dependent manner in resistant cells. DOX+AMR combination also inhibited P-gp to the same level as AMR at 4 μg/ml concentration.

In Examples 1-5 it is shown that AMR is a substrate for P-gp and it has chemosensitizing effect on, doxorubicin cytotoxicity. In both multidrug resistant leukemia (CEM/VLB) and colon carcinoma (SW620/Ad-300) cell lines, AMR and DOX combination showed modulation of DOX cytotoxicity. If Modulator Index (MI=fold decrease in resistance/modulator in μM concentration) is used to represent the effectiveness of an efflux blocker as proposed by Beck and Qian (Beck, W. T. and Qian, X. Biochem Pharmacol, 1992, 43:89-93), AMR has a MI of 40 in CEM/VLB and 41.6 in SW620/Ad-300 cell lines. This MI is much higher than that of most other efflux blockers such as verapamil and chlorpromazine in the vinblastine resistant human leukemia cell lines (Zamora, J. M. et al. Mol Pharmacol, 1998, 33:454-462; Pearce, H. L. et al Adv Enzyme Regul, 1990, 30:357-373).

Flow cytometric analysis of DOX accumulation in the presence of AMR showed that AMR enhanced DOX accumulation in multidrug resistant CEM/VLB and SW620/AD-300 cell lines. Cellular DOX accumulation was higher with 2 μg/ml of AMR than with 1 μg/ml AMR. AMR also inhibits [³H]-azidopine binding of P-gp in a dose-dependent manner in the photolabeling experiments. Even 2 μg/ml AMR was quite enough to inhibit [³H]-azidopine binding in both CEM/VLB and SW620/Ad-300 cells. Based on the effect of AMR on DOX accumulation and cytotoxicity, and also on the basis of inhibition of [³H]-azidopine binding of P-gp, we report that the AMR is a substrate for P-gp. AMR can be combined with doxorubicin as it may compete for closely related binding sites on P-gp in multidrug resistant cells.

AMR as a single agent at high concentration (>2.5 μg/ml) is also cytotoxic in several tumor cell lines. AMR causes cell cycle perturbation in leukemia and colon carcinoma cell lines when treated with AMR for 48 hours. AMR treatment induced a G₂+M arrest in all cell lines irrespective of their sensitivity to anthracyclines. The percentage of cells in G₂+M phase increased in a dose-dependent manner between 0-2.5 μg/ml of AMR. Furthermore, as described in detail in Examples 6-8, it has been observed that AMR causes caspase activation, G₂+M phase-arrest, and apoptosis in breast carcinoma cells.

### Example 6

### Cytotoxicity of AMR

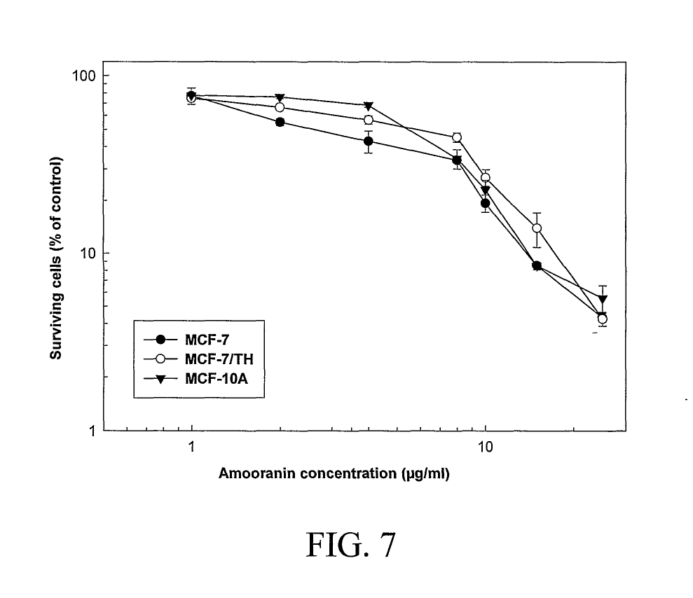

The IC₅₀ values were determined by MTT assay after exposing MCF-7, MCF-7/TH and MCF-10A cells to 0-8 μg/ml AMR for 48 hours. AMR dose-response curves of cell lines are given in FIG. 7. The results have indicated that AMR is cytotoxic to breast cancer cells. MCF-7, MCF-7/TH and MCF-10A cells incubated with AMR demonstrated a dose-dependent reduction of cell viability that reached maximal effects at 8 μg/ml (FIG. 7). MCF-7 cell line was more sensitive to AMR (IC₅₀=3.8 μg/ml) than MCF-7/TH (IC₅₀=6.8 μg/ml) and MCF-10A (IC₅₀=6.9 μg/ml) cell lines (p=0.0004).

### Example 7

### AMR Induces Cell Cycle Arrest

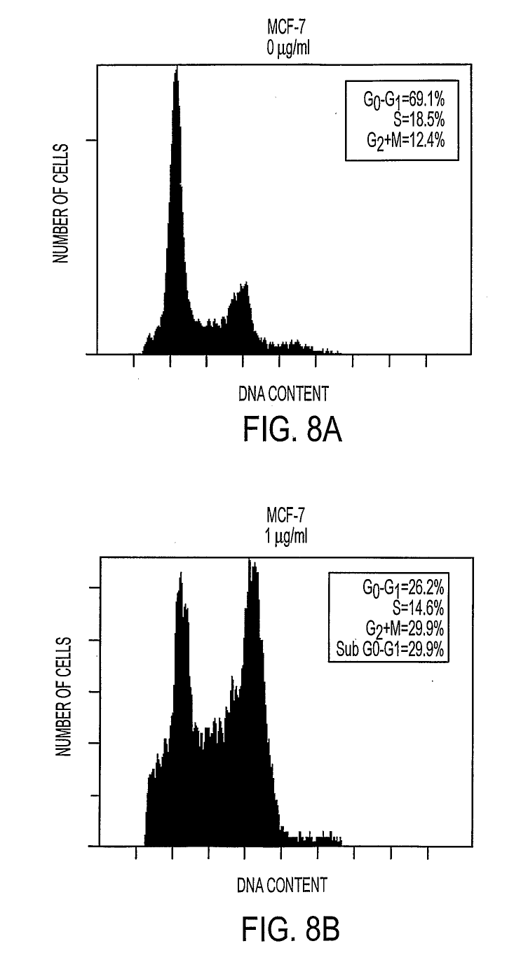

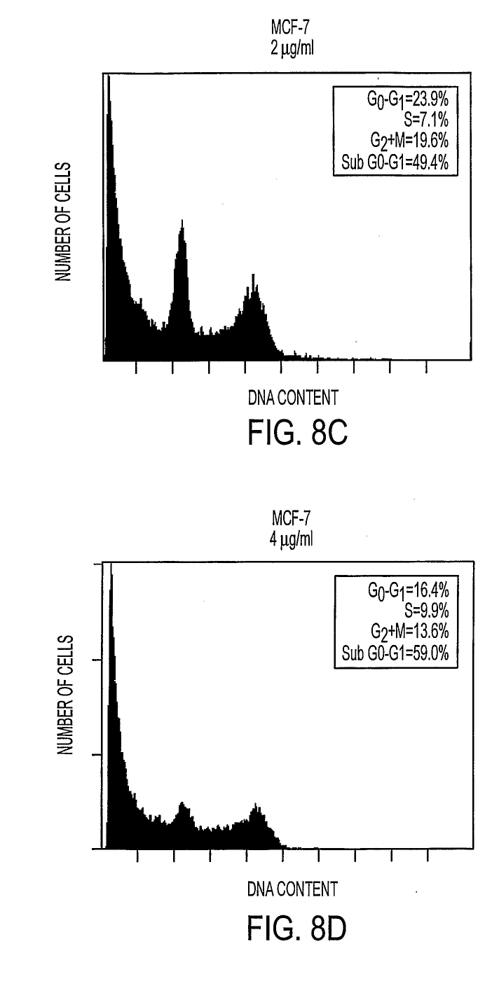

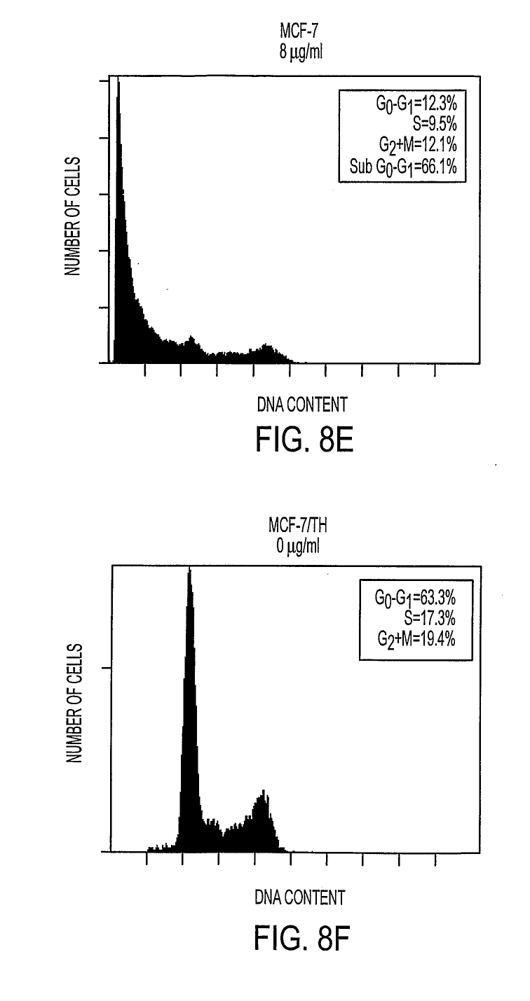

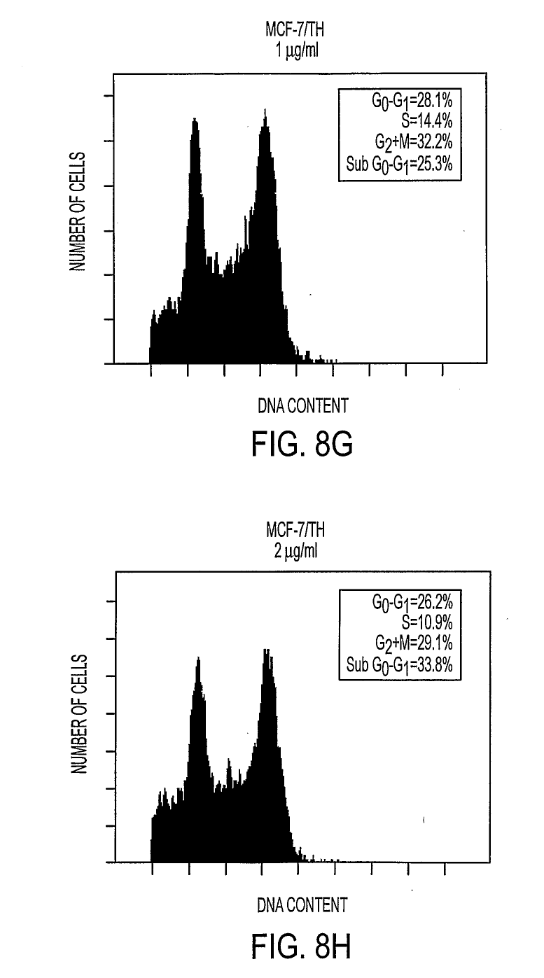

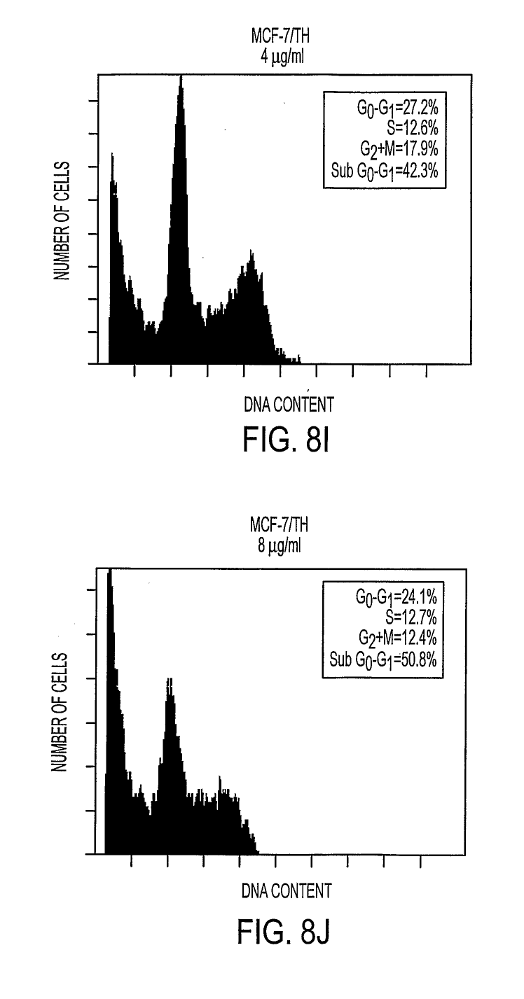

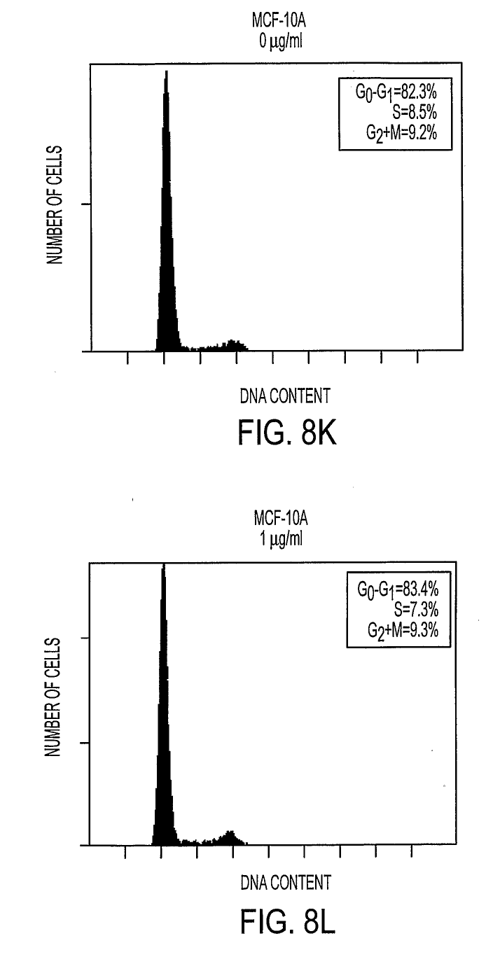

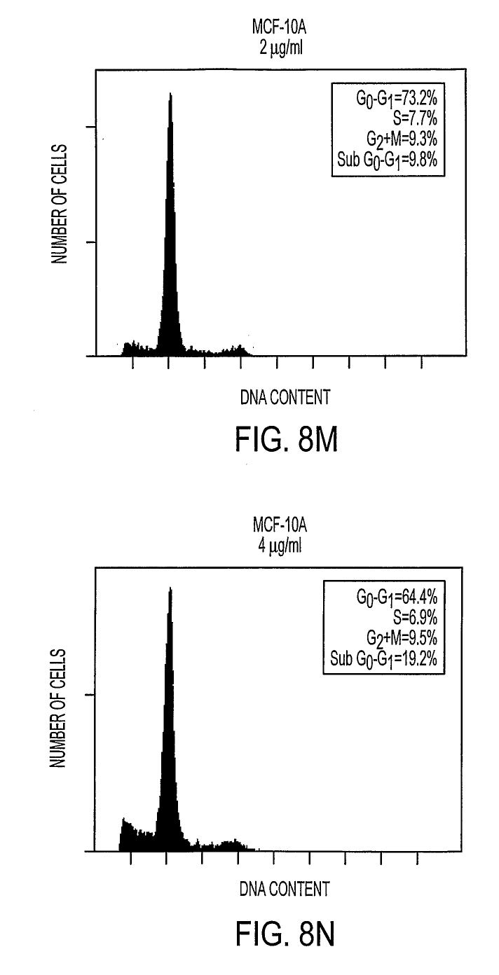

MCF-7 and MCF-7/TH cells treated with 1 μg/ml AMR showed accumulation in G₂+M phase population (12.4%-32.2%) with a concomitant decrease in G₀+G₁ phase cells suggesting a possible cell cycle arrest at G₂+M phase (FIG. 8). However, 1 μg/ml AMR does not induce any G₂/M phase arrest in MCF-10A cells. AMR also induced a higher percentage of sub G₀-G₁ population, indicative of apoptosis, in both breast cancer cell lines (MCF-7 and MCF-7/TH) than human breast epithelial cell line (MCF-10A) at every concentration of drug tested in the present investigation (FIG. 8).

### Example 8

### AMR-Induced Apoptosis

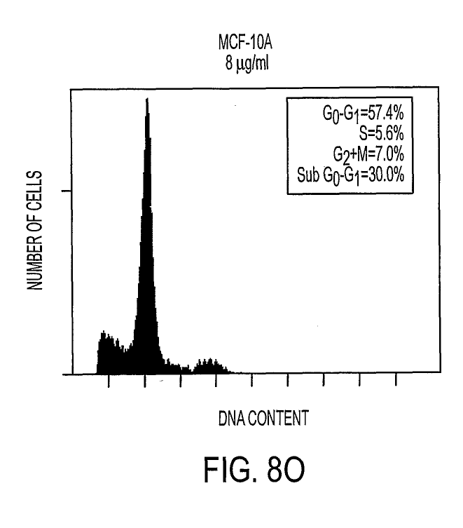

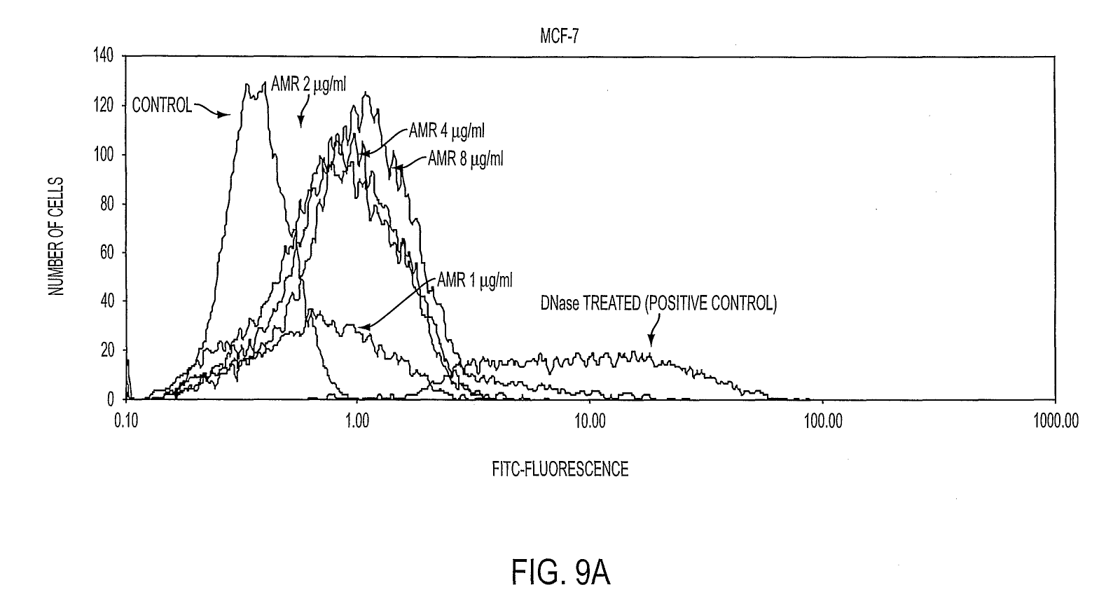

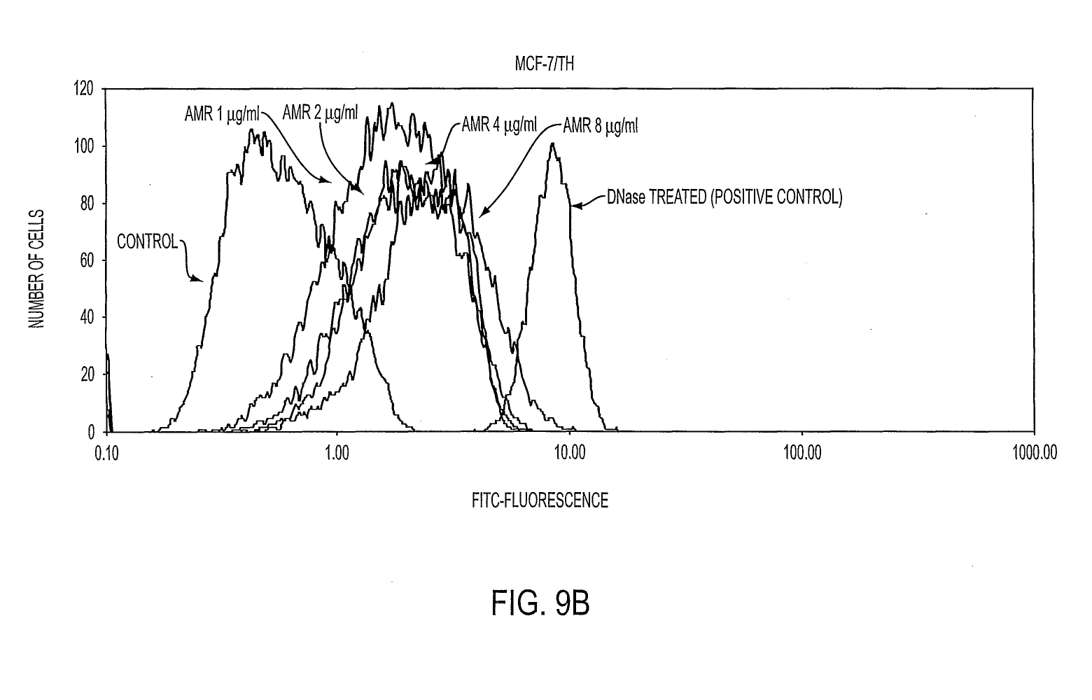

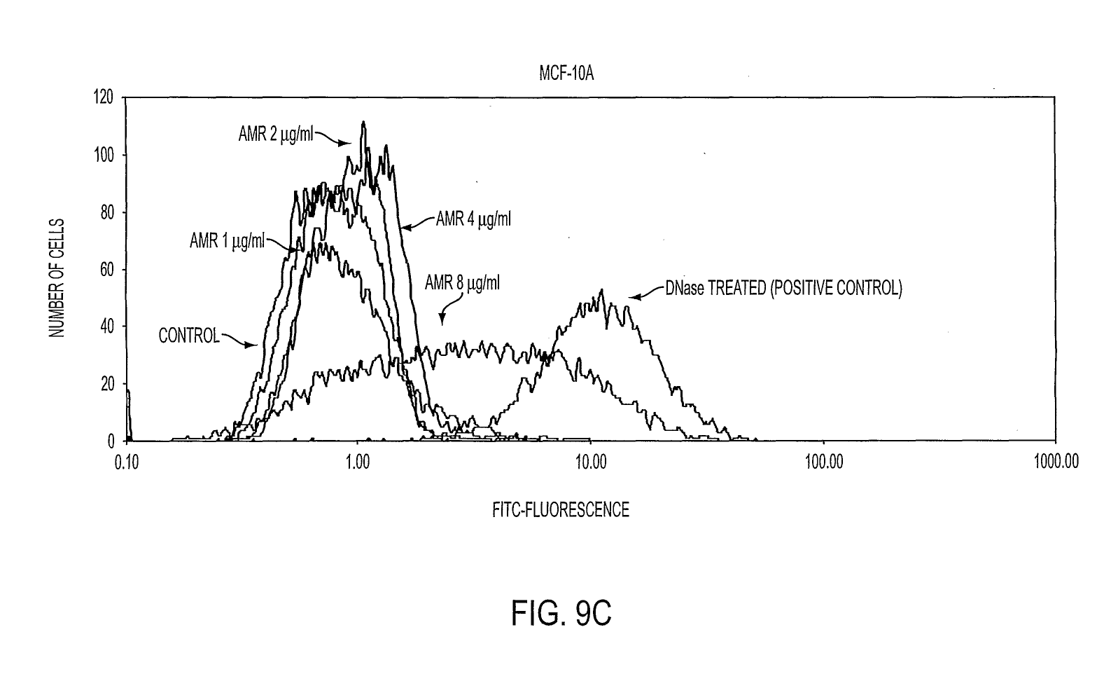

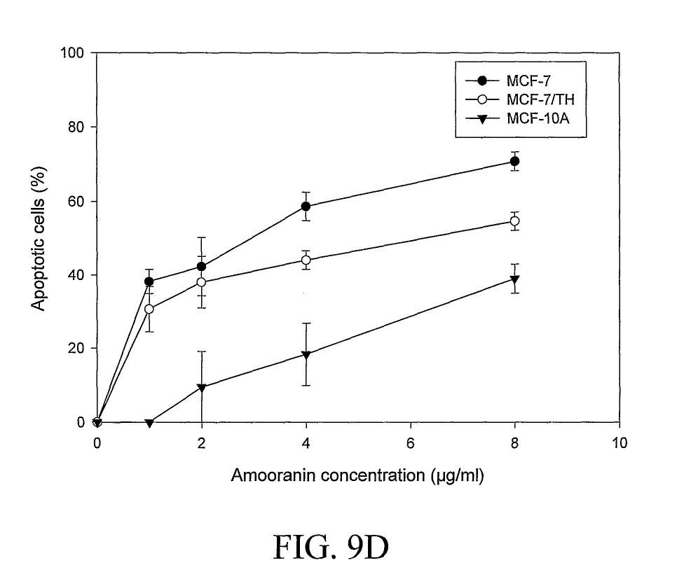

Apoptosis induced by AMR was investigated by different techniques such as DNA fragmentation, general caspase activity and proteolytic activation of caspase-8. The results of the TUNEL assay are shown in FIGS. 9A-9D. AMR caused DNA fragmentation in 37.3-72.1% MCF-7 cells, 32-48.7% MCF-7/TH cells, as compared with 0-37.1% MCF-10A cells at 1-8 μg/ml concentrations. DNase-treated positive control samples showed more than 95% apoptotic cells in all three cell lines. Drug sensitive (MCF-7) and multidrug resistant (MCF-7/TH) cell lines were more sensitive to AMR than breast epithelial cell line (MCF-10A) with respect to induction of apoptosis. Furthermore, the MCF-7 cell line was more sensitive to AMR than MCF-7/TH.

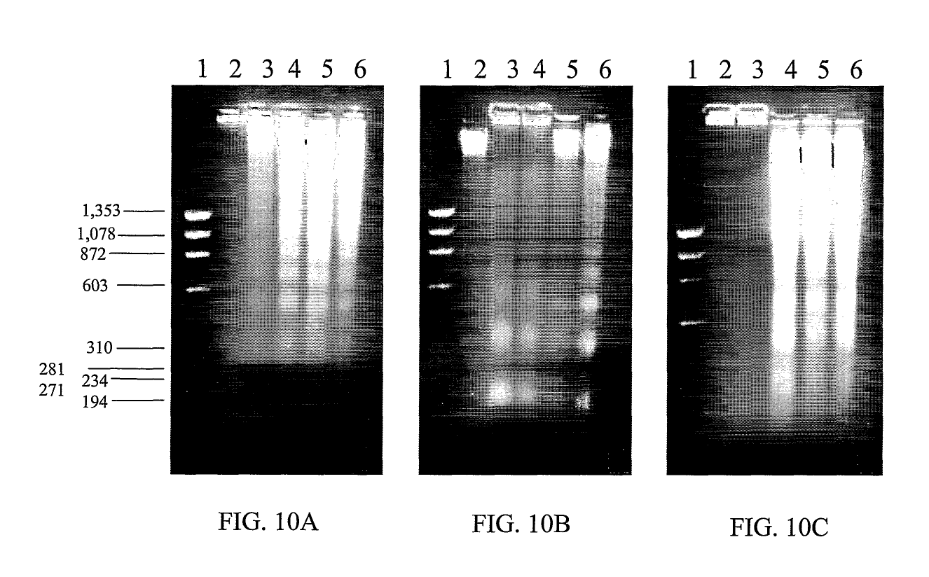

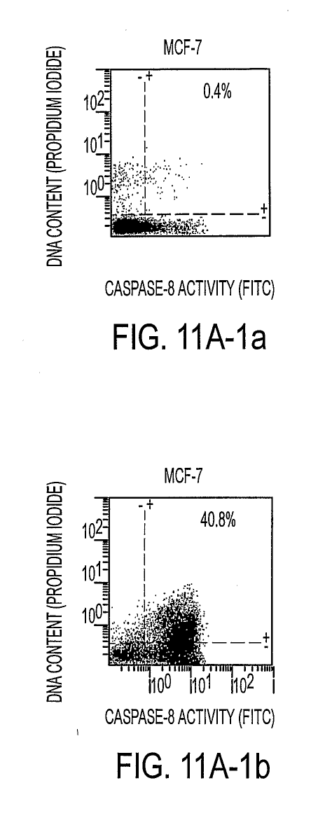

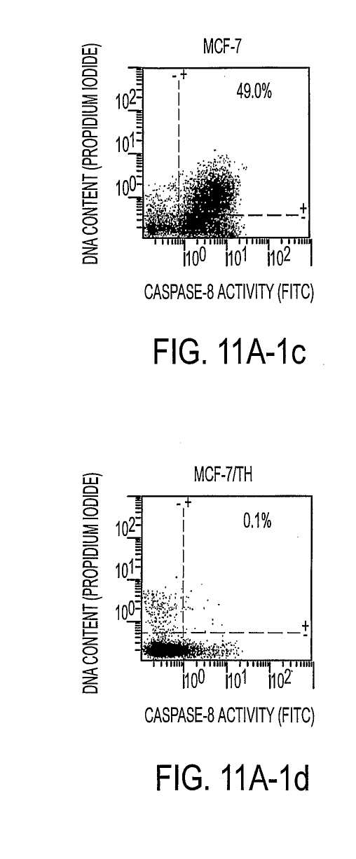

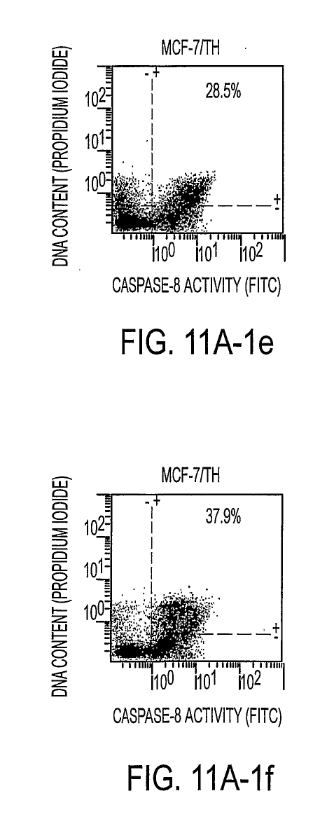

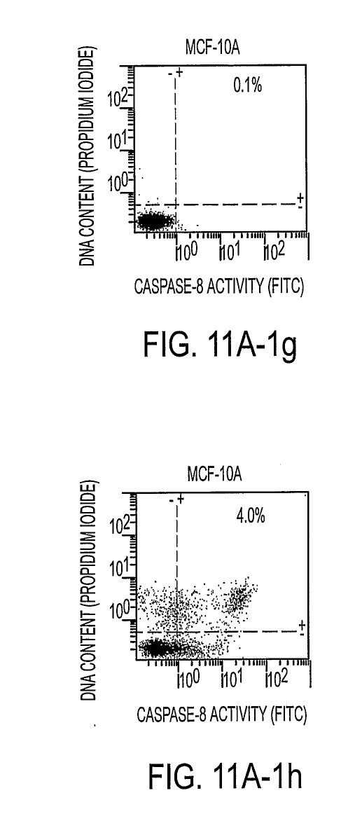

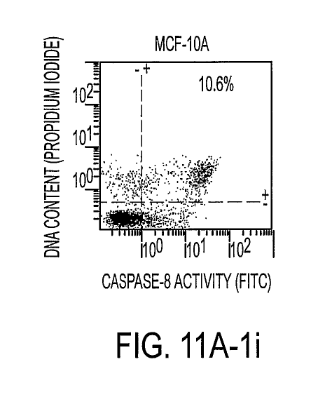

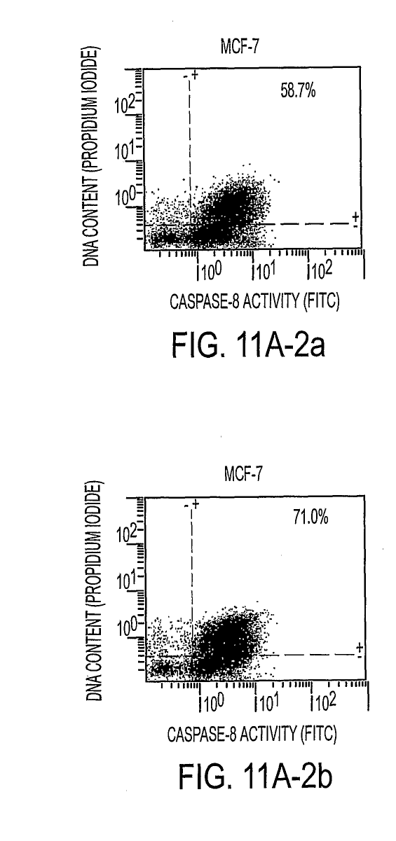

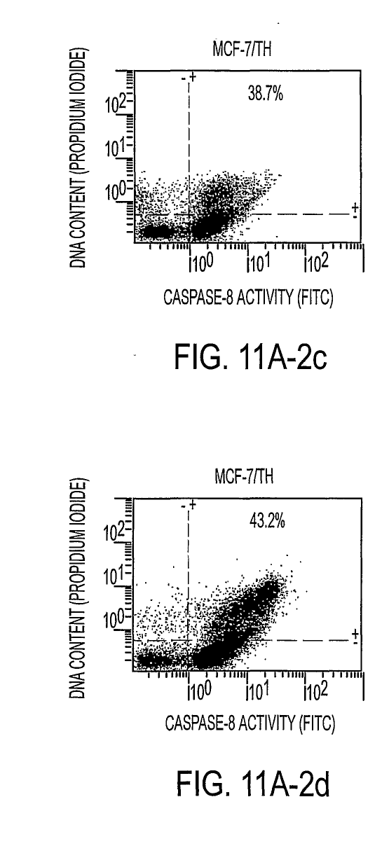

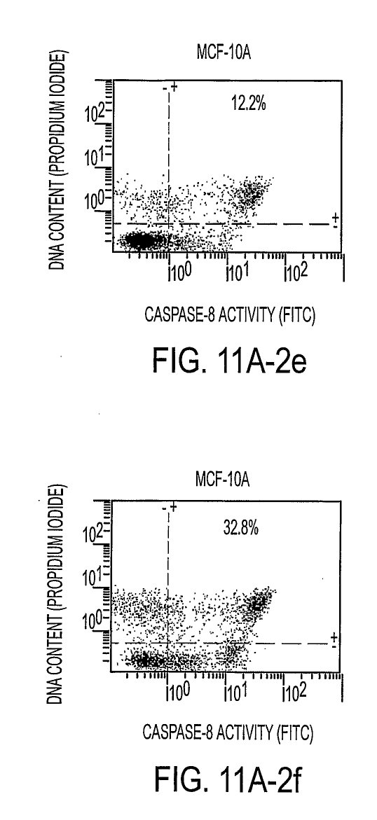

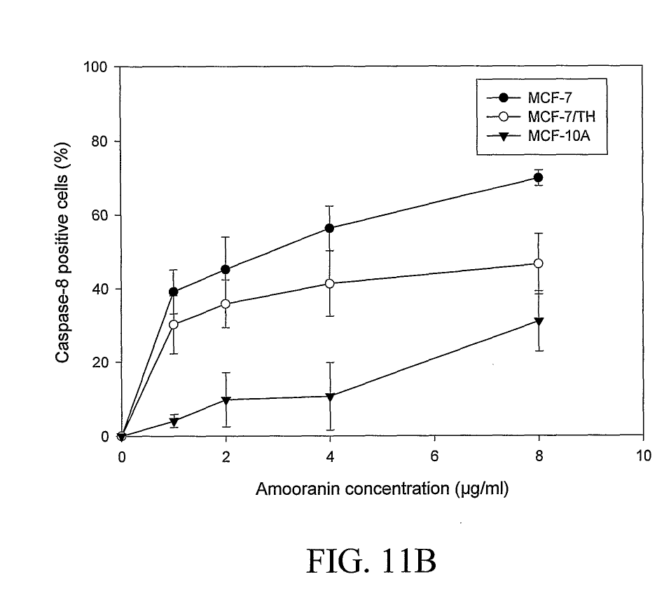

Analysis of internucleosomal DNA fragmentation induced by AMR in MCF-7, MCF-7/TH and MCF-10A cells was detected by gel electrophoresis of low molecular weight DNA (FIGS. 10A-10C). AMR has induced characteristic DNA ladder pattern, a hallmark of apoptosis, when cells were treated for 48 hours. AMR induced distinct ladder pattern in both cancer cell lines (MCF-7 and MCF-7/TH), although the ladder was less distinct in human breast epithelial cell lines (MCF-10A). Moreover, DNA fragmentation was almost undetectable in MCF-10A cells treated with 1 μg/ml AMR for 48 hours, whereas at the same concentration ladder pattern was highly visible in MCF-7 and MCF-7/TH cell lines.

To analyze the mechanism of AMR-induced apoptosis, activation of general caspase was examined. General caspase activity was generally higher in AMR-treated MCF-7 and MCF-7/TH cell lines than MCF-10A cell line (data not shown). To characterize the specific caspase further, activation of initiator caspase-8, a key component of caspase, was analyzed. Both MCF-7 and MCF-7/TH breast cancer cell lines showed higher percentage of caspase-8 positive cells at 1-8 μg/ml AMR than MCF-10A human breast epithelial cell line (FIGS. 6A and 6B). AMR caused caspase activation in 40.8-71% MCF-7 cells, 28.5-43.2% MCF-7/TH cells as compared with only 4-32.8% MCF-10A cells at 1-8 μg/ml concentrations.

As described in Examples 6-8, it was determined that AMR has a more potent effect on inhibiting the growth of MCF-7 breast cancer cells as compared with that on multidrug-resistant MCF-7/TH breast cancer cells and MCF-10A breast epithelial cells. MCF-7/TH is a MDR cell line developed from MCF-7 by repetitive exposure to adriamycin and it has an active P-glycoprotein pump. It has also been showed that AMR is a substrate for P-glycoprotein and AMR can increase adriamycin accumulation in tumor. Therefore a higher AMR IC₅₀ for MCF-7/TH than MCF-7 cell line can be expected. However, the reason for higher AMR IC₅₀ value of MCF-10A cells is not clear other than its almost normal cell phenotype.

AMR induces accumulation of cells at G₂+M phase during cell cycle traverse at 1 μg/ml. Alteration in the cell cycle phase distribution by AMR is encouraging for the sensitivity of tumor cells to DNA targeting agents. AMR is a triterpene acid having a similar basic chemical structure as other plant terpenoids like oleanolic acid (Suh, N. et al. Cancer Res, 1999, 59:336-341).

Induction of apoptosis appears to be the primary mechanism of AMR induced cell death, as shown by propidium iodide staining, DNA fragmentation and TUNEL labeling. Breast cancer cells exhibited discrete DNA fragmentation patterns as being associated with apoptosis. AMR is more specific to tumor cells, since apoptotic cell death induced by AMR in normal mammary epithelial MCF-10A cells is significantly lower than in breast cancer cells.

Several reports have demonstrated that the caspase family plays an important role in apoptosis (Patel, T. et al. FASEB J, 1997, 10:587-597; Zhivotovsky, B. et al. Biochem Biophys Res Commun, 1997, 230:481-488; Cohen, G. M. Biochem J, 1997, 326:1-16; Nicholson, D. W. et al. Nature, 1995, 376:37-43; Rao, L. and White, E. Curr Opin Gene Dev, 1997, 7:52-58; Salvesen, G. S, and Dixit, V. M. Cell, 1997, 91:443-446). In the present study, the results also show that AMR causes caspase-8 activation during the apoptotic process in a dose-dependent manner. The breast cancer cells have shown higher caspase-8 activity after 48 hours of AMR treatment than MCF-10A breast epithelial cells. Of the 14 caspases cloned to date, caspase-8 has been grouped under apical caspases along with caspase 2, 9 and 10. These apical caspases are reported to activate effector caspases 3, 6 and 7 (Thomberry, N. A. and Lazebnik, Y. Science, 1998, 281:1312-1316; Hu, S. et al. J Biol Chem, 1998, 273:29648-29653). MCF-7 cells have a deletion mutation on exon 3 of the caspase-3 gene (Janicke, R. U. et al. J Biol Chem, 1998, 273:9357-9360). Recombinant caspase-8 is able to process/activate all known caspases, including caspase-1 to −7 and caspase-9 and -10 (Fernandes, A. T. et al. Proc Natl Acad Sci USA, 1996, 93:7464-7469) and it lies at the apex of the apoptotic cascade (Boldin, M. P. et al. Cell, 1996, 85:803-815). The importance of the FADD-like prodomains of caspase-8 in directly linking CD95 and TNFR-1 mediated apoptosis has already been emphasized (Boldin, M. P. et al. Cell, 1996, 85:803-815). Perhaps AMR treatment may enable the prodomains of caspase-8 to be recruited specifically to facilitate the apoptosis. AMR induction of apoptosis by activation of caspase-8 in breast carcinoma cells can be expected and corroborates the effects already reported with synthetic triterpenoids like CDDO and CDDO-Me. Bedner et al reported a correlation between the apoptotic index estimated by the presence of DNA strand breaks (TUNEL assay) and the activation of caspases in cultures treated with anticancer agents (Bedner, E. et al. Exp Cell Res, 2000, 259:308-313). A strong correlation between levels of caspases and DNA strand breaks (as determined by TUNEL assay) has been observed in cells treated with AMR for 48 hours (r=0.95, p<0.05). This suggests that the estimates of frequency of cells undergoing apoptosis in the cell systems described in this study are almost similar whether it is based on caspases activation, or DNA fragmentation (TUNEL assay). In conclusion, it has been demonstrated that AMR induces cell cycle arrest, subsequently inducing apoptosis in MCF-7, MCF-7/TH and MCF-10A cells indicating the anticancer effect and clinical potential of this natural triterpene compound.

### Example 9

### Evaluation of Antitumor Activity of AMR in Human Tumor Xenografts

An evaluation of antitumor activity of AMR in human tumor xenografts was carried out in nude mice as described in the Material and Methods section. Preliminary xenograft studies with AMR showed that AMR has in vivo anti-tumor effects.

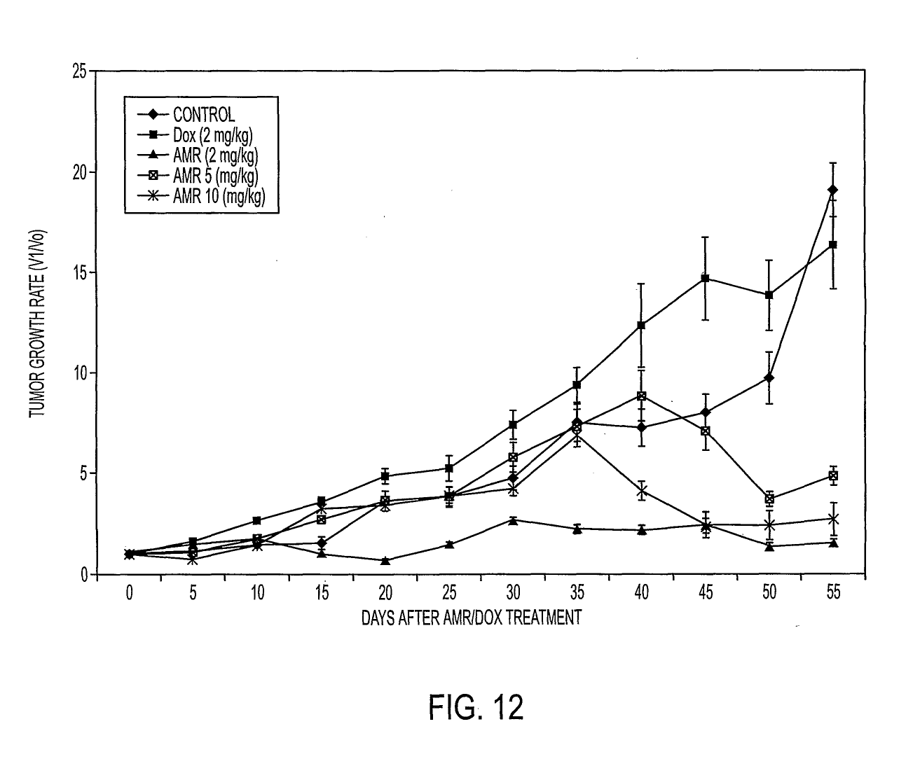

The effect of intraperitoneal infection of AMR on tumor growth rate in SW20 xenografts is shown in FIG. 12. AMR at 2-10 mg/kg significantly reduced tumor growth rate compared to doxorubicin (2 mg/kg) or saline treatments. AMR at 2 mg/kg was more effective than higher doses in reducing tumor growth rate. The superiority of AMR treatment over doxorubicin is also indicated by the high therapeutic value (Table 2). AMR therapy also resulted in better survival rates than saline treatment and the 2 mg/kg dose was more effective than higher doses (Table 3).

TABLE 2
Therapeutic Index of Amooranin
in SW620 human tumor xenografts

| Treatment | Therapeutic ratio |
|---|---|
| Control (saline) | 0 |
| Dox (2 mg/kg) | 26.6 |
| AMR (2 mg/kg) | 61.3** |
| AMR (5 mg/kg) | 37.6** |
| AMR (10 mg/kg) | 41.9** |

### Therapeutic Ratio=100—Tumor Growth Rate Index (GRI)

$$Where GR Index = \frac{\begin{matrix}Tumor growth rate \\ of a treated mouse\end{matrix}}{\begin{matrix}Average tumor growth \\ rate of control mouse\end{matrix}} \times 100$$

TABLE 3
Survival in Amooranin-treated SW620 human tumor xenografts

| Treatment | Survival (%) |
|---|---|
| Control (saline) | 33.3 |
| Doxorubicin (2 mg/kg) | 83.3 |
| AMR (2 mg/kg) | 83.3 |
| AMR (5 mg/kg) | 57.1 |
| AMR (10 mg/kg) | 57.1 |

Microarray hybridization of tumor RNAs from xenografts on AGILENT 22K human microarrays (22000 genes) (AGILENT TECHNOLOGIES, Palo Alto, Calif.) showed that 147 genes were up-regulated (>3-fold) and 57 genes down-regulated (<3-fold) at all AMR doses. AMR specifically upregulated several genes involved in drug transport, cell signaling, cytochrome c release, apoptosis, G2/M cell cycle arrest and inflammatory response. AMR also down-regulates genes involved in angiogenesis and immune response.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. An isolated compound having the following chemical structure (II) or a pharmaceutically acceptable salt thereof: 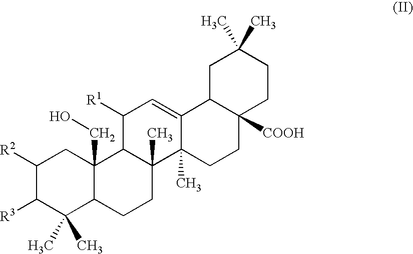
wherein R¹, R², and R³ may be the same or different, and are each selected from the group consisting of H, O, CN, CH₃COO, alkyl, alkenyl, alkynyl, halogen, and alkoxy, and wherein the isolated compound is not amooranin (25-hydroxy-3-oxoolean-12-en-28-oic acid).

2. The isolated compound of claim 1, wherein said compound has a chemical structure selected from the group consisting of (III), (TV), (V), (VI), (VII), (VIIT), (IX), (X), and (XI), or a pharmaceutically acceptable salt thereof:

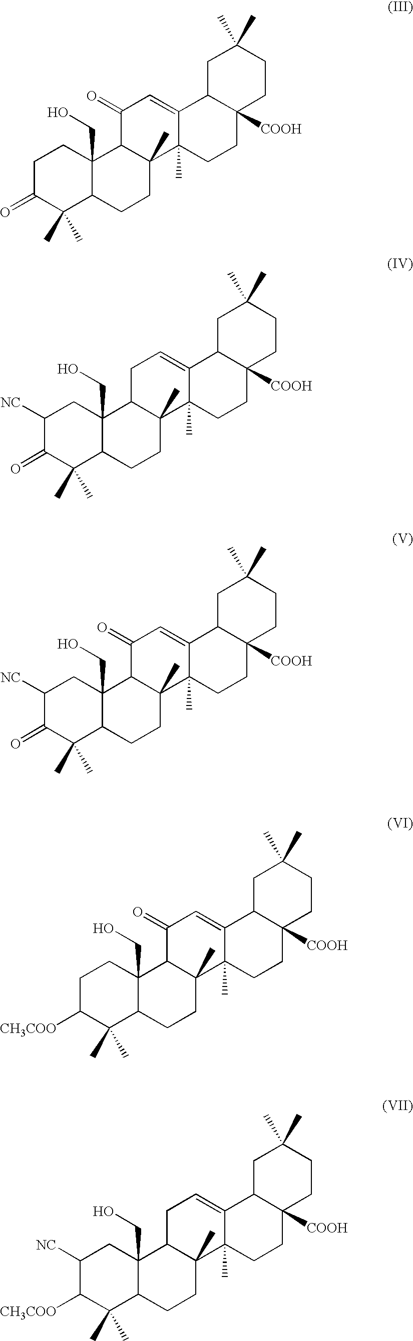

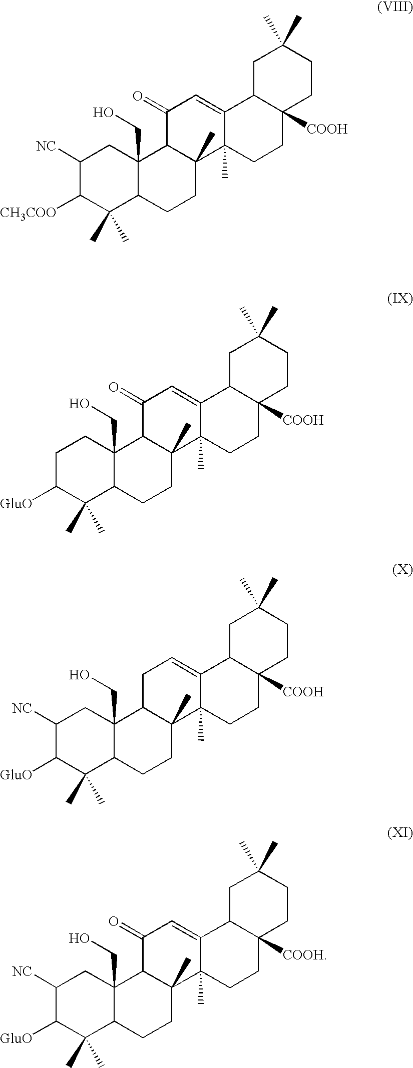

3. A pharmaceutical composition comprising an isolated compound having the following chemical structure (II), or a pharmaceutically acceptable salt thereof; and a pharmaceutically acceptable carrier: 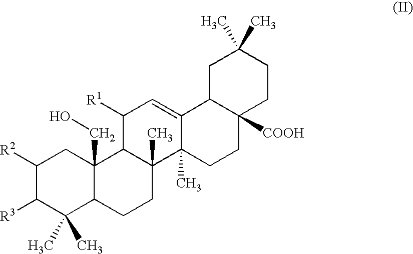
wherein R¹, R², and R³ may be the same or different, and are each selected from the group consisting of H, O CN, CH₃COO, alkyl alkenyl, alkynyl, halogen, and alkoxy, and wherein the isolated compound is not amooranin (25-hydroxy-3-oxoolean-12-en-28oic acid).

4. The pharmaceutical composition of claim 3, wherein said compound has a chemical structure selected from the group consisting of (III), (IV), (V), (VI), (VII), (VIII), (IX), (X), and (XI), or a pharmaceutically acceptable salt thereof:

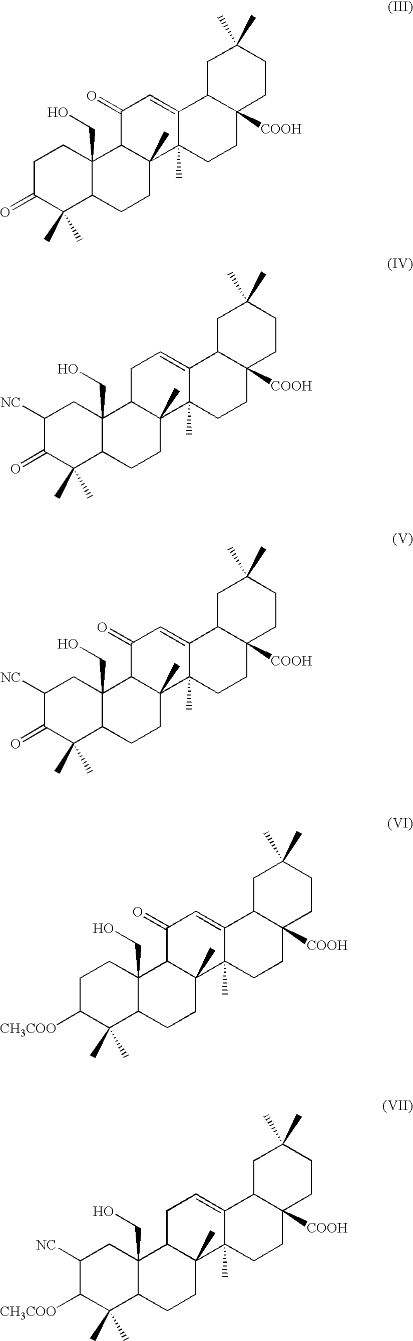

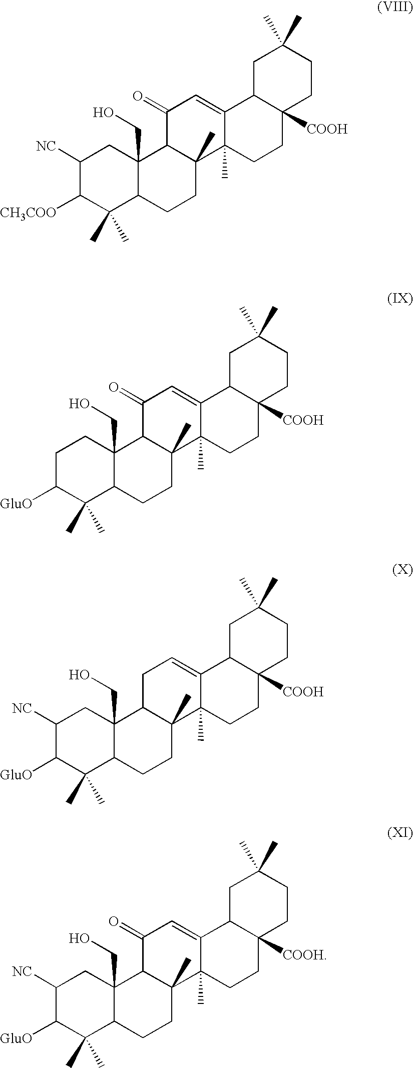

5. The pharmaceutical composition of claim 3, wherein said composition further comprises at least one anti-cancer agent.

6. A method for reducing proliferation in a target cell, comprising contacting a target cell with an effective amount of an amooranin analog, wherein the amooranin analog has the following chemical structure (II), or a pharmaceutically acceptable salt thereof: 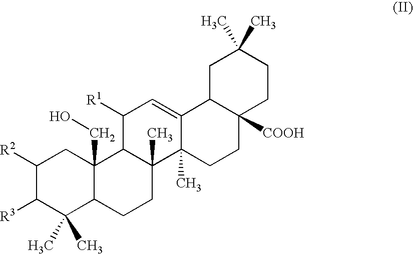
wherein R¹, R², and R may be the same or different, and are each selected from the group consisting of II, O, CN, CH₃COO, alkyl, alkenyl, alkynyl, halogen, and alkoxy, and wherein chemical structure (II) does not include amooranin (25-hydroxy-3-oxoolean-12-en-28-oic acid).

7. The method of claim 6, wherein the amooranin analog has a chemical structure selected from the group consisting of (III), (IV), (V), (VI), (VII), (VIII), (IX), (X), and (XI), or a pharmaceutically acceptable salt thereof:

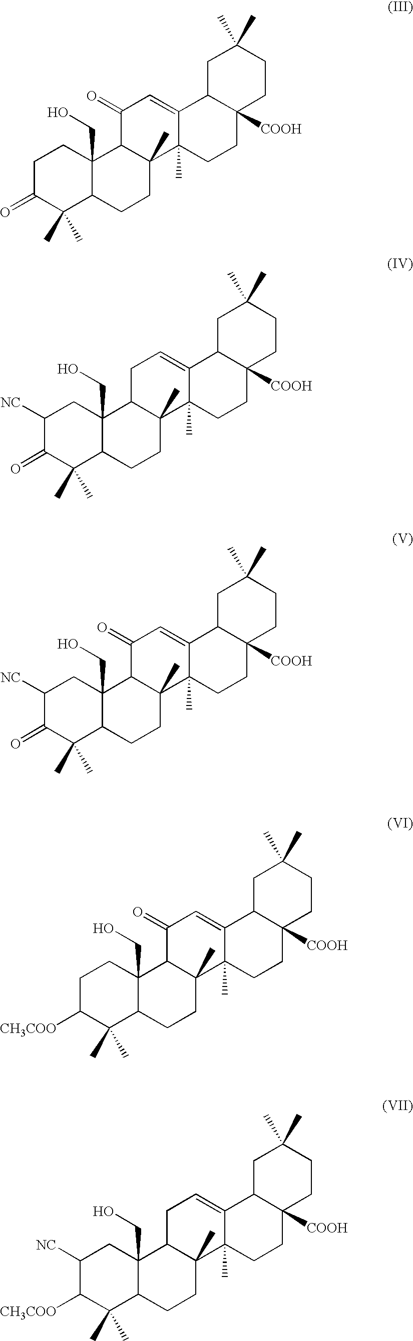

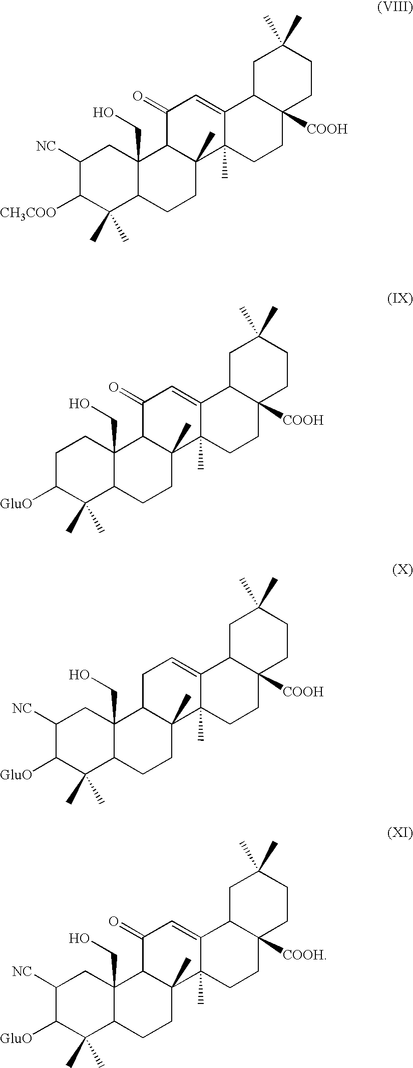

8. The method of claim 6, wherein said contacting is carried out in vitro.

9. The method of claim 8, wherein said contacting is carried out in vitro, and wherein the target cell is that of a tumor cell line.

10. The method of claim 8, wherein the target cell is a drug-sensitive cancer cell or multi-drug resistant (MDR) cancer cell.

11. The method of claim 6, wherein said contacting is carried out in vivo, and wherein said contacting comprises administering the amooranin analog to a patient.

12. The method of claim 11, wherein the patient is suffering from a proliferative disorder characterized by unregulated cell growth, and wherein the target cell is at a site of unregulated cell growth.

13. The method of claim 12, wherein the proliferative disorder is cancer.

14. The method of claim 13, wherein the proliferative disorder is selected from the group consisting of carcinoma, lymphoma, blastoma, sarcoma, and leukemia.

15. The method of claim 13, wherein the proliferative disorder is selected from the group consisting of breast cancer, prostate cancer, colon cancer, squamous cell cancer, small-cell lung cancer, non-small cell lung cancer, gastrointestinal cancer, pancreatic cancer, cervical cancer, ovarian cancer, liver cancer, bladder cancer, colorectal cancer, endometrial carcinoma, kidney cancer, and thyroid cancer.

16. The method of claim 13, wherein the proliferative disorder is selected from the group consisting of basal cell carcinoma, biliary tract cancer; bone cancer; cancer of the central nervous system (CNS); choriocarcinoma; connective tissue cancer; esophageal cancer; eye cancer; cancer of the head and neck; gastric cancer; intra-epithelial neoplasm; larynx cancer; lymphoma; melanoma; myeloma; neuroblastoma; oral cavity cancer; pancreatic cancer; retinoblastoma; rhabdomyosarcoma; rectal cancer; cancer of the respiratory system; sarcoma; skin cancer; stomach cancer; testicular cancer; uterine cancer; and cancer of the urinary system.

17. The method of claim 13, wherein the amooranin analog is administered to the patient systemically.

18. The method of claim 11, wherein the amooranin analog is administered to the target cell locally.

19. The method of claim 6, wherein the target cell is a human cell.

20. The method of claim 6, wherein the amooranin analog induces apoptosis of the target cell.

21. The method of claim 6, wherein the method further comprises contacting the target cell with an anti-cancer agent, wherein the target cell is multi-drug resistant (MDR), and wherein the amooranin analog makes the target cell more sensitive to the anti-cancer agent.

22. The method of claim 11, wherein the target cell is a benign or malignant tumor cell at a tumor site in the patient, and wherein the amooranin analog reduces the size of the tumor.

23-26. (canceled)
